(19) United States
(12) Patent Application Publication
(10) Pub. No.: US 2016/0082129 A1
(43) Pub. Date: Mar. 24, 2016

(54) VE-PTP Extracellular Domain Antibodies Delivered by a Gene Therapy Vector

(71) Applicant: Aerpio Therapeutics, Inc., Cincinnati, OH (US)

(72) Inventor: Kevin Peters, Cincinnati, OH (US)

(21) Appl. No.: 14/862,948

(22) Filed: Sep. 23, 2015

Related U.S. Application Data
(60) Provisional application No. 62/054,752, filed on Sep. 24, 2014.

(51) Int. Cl. A61K 48/00; A61K 39/395; C12N 15/86

(52) U.S. Cl. CPC A61K 48/00; C12N 15/86; A61K 39/3955; C12N 2750/14143; A61K 2039/505

(57) ABSTRACT

The disclosure provides compositions and methods for the treatment of ocular conditions associated with angiogenesis, comprising administering a nucleic acid that encodes for a tyrosine phosphatase suppressor to a subject.

### CROSS REFERENCE

This application claims the benefit of U.S. Provisional Application No. 62/054,752, filed Sep. 24, 2014, the entire content of which is incorporated herein by reference in its entirety.

### BACKGROUND

The eye comprises several structurally and functionally distinct vascular beds that supply ocular components critical to the maintenance of vision. These beds include the retinal and choroidal vasculatures, which supply the inner and outer portions of the retina, respectively, and the limbal vasculature located at the periphery of the cornea. Injuries and diseases that impair the normal structure or function of these vascular beds are among the leading causes of visual impairment and blindness. For example, diabetic retinopathy is the most common disease affecting the retinal vasculature, and is the leading cause of vision loss among the working age population in the United States. Vascularization of the cornea secondary to injury or disease is yet another category of ocular vascular disease that can lead to severe impairment of vision.

### SUMMARY OF THE INVENTION

In some embodiments, the invention provides a pharmaceutical composition comprising a nucleic acid, wherein the nucleic acid is carried by a vector, wherein the nucleic acid encodes a tyrosine phosphatase suppressor.

In some embodiments, the invention provides a pharmaceutical composition comprising a nucleic acid, wherein the nucleic acid is carried by a vector, wherein the nucleic acid encodes a Tie2 activator.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 depicts a schematic representation of an illustrative therapeutic nucleic acid of the disclosure.

FIG. 2 illustrates the reaction and product of an experiment of Example 1. The figure is a graphical representation of the mean area of choroidal neovascularization in C57BL/6 mice 14 days post laser injury in eyes treated with intravitreal injection of 1 μg or 2 μg of an anti-VE-PTP extracellular domain antibody in one eye versus similar treatment of the fellow eye with control.

FIG. 3 illustrates the reaction and product of an experiment of Example 1. The figure shows the mean area (mm²) of retinal neovascularization in C57BL/6 mice on day P17 after containment in a 75% oxygen atmosphere from P5 to P12 and intravitreal injection of an anti-VE-PTP extracellular domain antibody at P12 when the mice were returned to ambient air.

FIG. 4 illustrates the reaction and product of an experiment of Example 1. The figure shows representative fluorescent micrographs of mouse retinas in the oxygen-induced retinopathy model after intravitreal injection of vehicle or 2 μg of an anti-VE-PTP extracellular domain antibody.

FIG. 5 illustrates the PCR of several combinations of Ig variable domain primers of Example 3.

FIG. 6 shows the individual and consensus amino acid sequence results for the V_{H} region of RI5E6. CDRs are in bold and underlined.

FIG. 7 shows the individual and consensus amino acid sequence results for the V_{L} region of RI5E6. CDRs are in hold and underlined.

### DETAILED DESCRIPTION

The present disclosure provides compositions and methods for the delivery of a vector comprising a nucleic acid encoding a suppressor of Human Protein Tyrosine Phosphatase-beta (HPTPβ) for the treatment of ocular disorders that are characterized by, for example, vascular instability, vascular leakage, and neovascularization. A composition of the disclosure can activate Tie2 signaling by promoting protein phosphorylation, such as phosphorylation of the Tie2 protein.

Tie-2 (tyrosine kinase with immunoglobulin and epidermal growth factor homology domains 2) is a membrane receptor tyrosine kinase found almost exclusively in vascular endothelial cells.

The principle regulators of Tie-2 receptor phosphorylation are Angiopoietin-1 (Ang-1) and Angiopoietin-2 (Ang-2). Upon Ang-1 binding to Tie-2, the level of Tie-2 receptor phosphorylation increases. The duration of Tie-2 receptor phosphorylation is regulated by HPTPβ, which cleaves off the phosphate. Tie-2 receptor phosphorylation helps maintain endothelial cell proximity, therefore, the duration of Tie-2 receptor phosphorylation is an important determinant of endothelial cell proximity. For example, when severe inflammation occurs, the capillary endothelial cells separate, allowing proteins to enter the interstitial space. Separation of the capillary endothelial cells, and subsequent leak of proteins in the interstitial space, is known as vascular leak and can lead to dangerous hypotension (low blood pressure), edema, hemoconcentration, and hypoalbuminemia. Inhibition of HPTPβ leads to increased levels of Tie-2 receptor phosphorylation, a process that can maintain or restore capillary endothelial cell proximity.

### Human Protein Tyrosine Phosphatase-beta (HPTPβ) Binding Agent

The present disclosure provides administering a nucleic acid that encodes a suppressor of HPTPβ to a subject in need thereof. Illustrative examples of HPTPβ include amino acid sequence SEQ ID NO.: 13 and an example of a nucleic acid sequence encoding HPTPβ is cDNA sequence SEQ ID NO.: 14. Target sequences can have at least about 90% homology, at least about 91% homology, at least about 92% homology, at least about 93% homology, at least about 94% homology, at least about 95% homology, at least about 96% homology, at least about 97% homology, at least about 98% homology, at least about 99% homology, at least about 99.1% homology, at least about 99.2% homology, at least about 99.3% homology, at least about 99.4% homology, at least about 99.5% homology, at least about 99.6% homology, at least about 99.7% homology, at least about 99.8% homology, at least about 99.9% homology, at least about 99.91% homology, at least about 99.92% homology, at least about 99.93% homology, at least about 99.94% homology, at least about 99.95% homology, at least about 99.96% homology, at least about 99.97% homology, at least about 99.98% homology, or at least about 99.99% homology to a nucleic acid or amino acid sequence provided herein. Various methods and software programs can be used to determine the homology between two or more peptides or nucleic acids, such as NCBI BLAST, Clustal W, MAFFT, Clustal Omega, AlignMe, Praline, or another suitable method or algorithm

Table 1 provides non-limiting examples of peptide and nucleic acid sequences of the invention. SEQ ID NO.: 1-3 are the consensus (SEQ ID NO. 1) and individual (SEQ ID NOS. 2 and 3) V_{H} amino acid sequences. SEQ ID NO.: 4 and 5 are the consensus and individual V_{L} amino acid sequences. SEQ ID NO.: 6-8 are the V_{H} CDR amino acid sequences. SEQ ID NO.: 9-10 are two of the V_{L} CDR amino acid sequences. SEQ ID NO.: 11 and 12 are the V_{H} and V_{L} nucleic acid sequences, respectively. SEQ ID NO.: 13 and 14 are the HPTPβ amino acid and cDNA nucleic acid sequences, respectively. SEQ ID NO.: 15 is the VE-PTP amino acid sequence. SEQ ID NO.: 16 is the amino acid sequence of the first 8 fibronectin type III-like (FN3) repeats of VE-PTP. SEQ ID NO.: 17 is the amino acid sequence of the extracellular domain of HPTPβ. SEQ ID NO.: 18 is the amino acid sequence of the first FN3 repeat of HPTPβ.

TABLE 1
Sequences of the invention

| SEQ | |
|---|---|
| ID NO. | Sequence |
| 1 | EVQLVETGGGLVQPKGSMKLSCAASGFTFNANAMNWIRQ |
| | APGKGLEWVARIRTKSNNYATYYAGSVKDRFTISRDDAQ |
| | NMLYLQMNDLKTEDTAMYYCVRDYYGSSAWITYWGQGTL |
| | VTVSA |
| 2 | EVQLVETGGGLVQPKGSMILSCAASGFTFNANAMNWIRQ |
| | APGKGLEWVARIRTKSNNYATYYAGSVKDRFTISRDDAQ |
| | NMLYLQMNDLKTEDTAMYYCVRDYYGSSAWTTYWGQGTL |
| | VTVSA |
| 3 | EVQLVETGGGLAQPKGSMKLSCAASGFTFNANAMNWIRQ |
| | APGKGLEWVARIRTKSNNYATYYAGSVKDRFTISRDDAQ |
| | NMLYLQMNDLKTEDTAMYYCVRDYYGSSAWITYWGQGTL |
| | VTVSA |
| 4 | DIVMTQSHKFMSTSVGDRVSITCKASQHVGTAVAWYQQK |
| | PDQSPKQLIYWASTRHTGVPDRFTGSGSGTDFTLTISNV |
| | QSEDLADYFCQQYSSYPFTFGSGTKLEIK |
| 5 | DIVMTQSHKFMSTSVGDRVSITCKASQHVGTAVAWYQQK |
| | PDQSPKQLIYWASTRHTGVPDRFTGSGSGSDFTLTISNV |
| | QSEDLADYFCQQYSSYPFTFGSGTKLEIK |
| 6 | GFTFNANA |
| 7 | IRTKSNNYAT |
| 8 | VRDYYGSSAWITY |
| 9 | QHVGTA |
| 10 | QQYSSYPFT |
| 11 | GAGGTGCAGCTTGTTGAGACTGGTGGAGGATTGGTGCAG |
| | CCTAAAGGGTCAATGAAACTCTCATGTGCAGCCTCTGGA |
| | TTCACCTTCAATGCCAATGCCATGAACTGGATCCGCCAG |
| | GCTCCAGGAAAGGGTTTGGAATGGGTTGCTCGCATAAGA |
| | ACTAAAAGTAATAATTATGCAACATATTATGCCGGTTCG |
| | GTGAAAGACAGGTTCACCATCTCCAGAGATGATGCACAG |
| | AACATGCTCTATCTGCAAATGAACGACTTGAAAACTGAG |
| | GACACAGCCATGTATTACTGTGTGCGAGATTACTACGGT |
| | AGTAGCGCCTGGATTACTTACTGGGGCCAAGGGACTCTG |
| | GTCACTGTCTCTGCA |
| 12 | GACATTGTGATGACCCAGTCTCACAAATTCATGTCCACA |
| | TCAGTAGGAGACAGGGTCAGCATCACCTGCAAGGCCAGT |
| | CAGCATGTGGGTACTGCTGTAGCCTGGTATCAACAGAAA |
| | CCAGACCAATCTCCTAAACAACTGATTTACTGGGCATCC |
| | ACCCGGCACACTGGAGTCCCTGATCGCTTCACAGGCAGT |
| | GGATCTGGGACAGATTTCACTCTCACCATTAGTAATGTG |
| | CAGTCTGAAGACTTGGCAGATTATTTCTGTCAGCAATAC |
| | AGCAGTTATCCATTCACGTTCGGCTCGGGGACAAAGTTG |
| | GAAATAAAA |
| 13 | MLSHGAGLALWITLSLLQTGLAEPERCNFTLAESKASSH |
| | SVSIQWRILGSPCNFSLIYSSDTLGAALCPTFRIDNTTY |
| | GCNLQDLQAGTIYNFKIISLDEERTVVLQTDPLPPARFG |
| | VSKEKTTSTGLHVWWTPSSGKVTSYEVQLFDENNQKIQG |
| | VQIQESTSWNEYTFFNLTAGSKYNIAITAVSGGKRSFSV |
| | YTNGSTVPSPVKDIGISTKANSLLISWSHGSGNVERYRL |
| | MLMDKGILVHGGVVDKHATSYAFHGLSPGYLYNLTVMTE |
| | AAGLQNYRWKLVRTAPMEVSNLKVTNDGSLTSLKVKWQR |
| | PPGNVDSYNITLSHKGTIKESRVLAPWITETHFKELVPG |
| | RLYQVTVSCVSGELSAQKMAVGRTFPDKVANLEANNNGR |
| | MRSLVVSWSPPAGDWEQYRILLFNDSVVLLNITVGKEET |
| | QYVMDDTGLVPGRQYEVEVIVESGNLKNSERCQGRTVPL |
| | AVLQLRVKHANETSLSIMWQTPVAEWEKYIISLADRDLL |
| | LIHKSLSKDAKEFTFTDLVPGRKYMATVTSISGDLKNSS |
| | SVKGRTVPAQVTDLHVANQGMTSSLFTNWTQAQGDVEFY |
| | QVLLIHENVVIKNESISSETSRYSFHSLKSGSLYSVVVT |
| | TVSGGISSRQVVVEGRTVPSSVSGVTVNNSGRNDYLSVS |
| | WLVAPGDVDNYEVTLSHDGKVVQSLVIAKSVRECSFSSL |
| | TPGRLYTVTITTRSGKYENHSFSQERTVPDKVQGVSVSN |
| | SARSDYLRVSWVHATGDFDHYEVTIKNKNNFIQTKSIPK |
| | SENECVFVQLVPGRLYSVTVTTKSGQYEANEQGNGRTIP |
| | EPVKDLTLRNRSTEDLHVTWSGANGDVDQYEIQLLFNDM |
| | KVFPPFHLVNTATEYRFTSLTPGRQYKILVLTISGDVQQ |
| | SAFIEGFTVPSAVKNIHISPNGATDSLTVNWTPGGGDVD |
| | SYTVSAFRHSQKVDSQTIPKHVFEHTFHRLEAGEQYQIM |
| | IASVSGSLKNQINVVGRTVPASVQGVIADNAYSSYSLIV |
| | SWQKAAGVAERYDILLLTENGILLRNTSEPATTKQHKFE |
| | DLTPGKKYKIQILTVSGGLFSKEAQTEGRTVPAAVTDLR |
| | ITENSTRHLSFRWTASEGELSWYNIFLYNPDGNLQERAQ |
| | VDPLVQSFSFQNLLQGRMYKMVIVTHSGELSNESFIFGR |
| | TVPASVSHLRGSNRNTTDSLWFNWSPASGDFDFYELILY |
| | NPNGTKKENWKDKDLTEWRFQGLVPGRKYVLWVVTHSGD |
| | LSNKVTAESRTAPSPPSLMSFADIANTSLAITWKGPPDW |
| | TDYNDFELQWLPRDALTVFNPYNNRKSEGRIVYGLRPGR |
| | SYQFNVKTVSGDSWKTYSKPIFGSVRTKPDKIQNLHCRP |
| | QNSTAIACSWIPPDSDFDGYSIECRKMDTQEVEFSRKLE |
| | KEKSLLNIMMLVPHKRYLVSIKVQSAGMTSEVVEDSTIT |
| | MIDRPPPPPPHIRVNEKDVLISKSSINFTVNCSWFSDTN |
| | GAVKYFTVVVREADGSDELKPEQQHPLPSYLEYRHNASI |
| | RVYQTNYFASKCAENPNSNSKSFNIKLGAEMESLGGKRD |
| | PTQQKFCDGPLKPHTAYRISIRAFTQLFDEDLKEFTKPL |
| | YSDTFFSLPITTESEPLFGAIEGVSAGLFLIGMLVAVVA |
| | LLICRQKVSHGRERPSARLSIRRDRPLSVHLNLGQKGNR |
| | KTSCPIKINQFEGHFMKLQADSNYLLSKEYEELKDVGRN |
| | QSCDIALLPENRGKNRYNNILPYDATRVKLSNVDDDPCS |
| | DYINASYIPGNNFRREYIVTQGPLPGTKDDFWKMVWEQN |
| | VHNIVMVTQCVEKGRVKCDHYWPADQDSLYYGDLILQML |
| | SESVLPEWTIREFKICGEEQLDAHRLIRHFHYTVWPDHG |
| | VPETTQSLIQFVRTVRDYINRSPGAGPTVVHCSAGVGRT |
| | GTFIALDRILQQLDSKDSVDIYGAVHDLRLHRVHMVQTE |
| | CQYVYLHQCVRDVLRARKLRSEQENPLFPIYENVNPEYH |
| | RDPVYSRH |
| 14 | GTCTCCTCTGGATCTTAACTACTGAGCGCAATGCTGAGC |
| | CATGGAGCCGGGTTGGCCTTGTGGATCACACTGAGCCTG |
| | CTGCAGACTGGACTGGCGGAGCCAGAGAGATGTAACTTC |
| | ACCCTGGCGGAGTCCAAGGCCTCCAGCCATTCTGTGTCT |
| | ATCCAGTGGAGAATTTTGGGCTCACCCTGTAACTTTAGC |
| | CTCATCTATAGCAGTGACACCCTGGGGGCCGCGTTGTGC |
| | CCTACCTTTCGGATAGACAACACCACATACGGATGTAAC |
| | CTTCAAGATTTACAAGCAGGAACCATCTATAACTTCAAG |
| | ATTATTTCTCTGGATGAAGAGAGAACTGTGGTCTTGCAA |
| | ACAGATCCTTTACCTCCTGCTAGGTTTGGAGTCAGTAAA |
| | GAGAAGACGACTTCAACCGGCTTGCATGTTTGGTGGACT |
| | CCTTCTTCCGGAAAAGTCACCTCATATGAGGTGCAATTA |
| | TTTGATGAAAATAACCAAAAGATACAGGGGGTTCAAATT |
| | CAAGAAAGTACTTCATGGAATGAATACACTTTTTTCAAT |
| | CTCACTGCTGGTAGTAAATACAATATTGCCATCACAGCT |
| | GTTTCTGGAGGAAAACGTTCTTTTTCAGTTTATACCAAT |
| | GGATCAACAGTGCCATCTCCAGTGAAAGATATTGGTATT |
| | TCCACAAAAGCCAATTCTCTCCTGATTTCCTGGTCCCAT |
| | GGTTCTGGGAATGTGGAACGATACCGGCTGATGCTAATG |
| | GATAAAGGGATCCTAGTTCATGGCGGTGTTGTGGACAAA |
| | CATGCTACTTCCTATGCTTTTCACGGGCTGTCCCCTGGC |
| | TACCTCTACAACCTCACTGTTATGACTGAGGCTGCAGGG |
| | CTGCAAAACTACAGGTGGAAACTAGTCAGGACAGCCCCC |
| | ATGGAAGTCTCAAATCTGAAGGTGACAAATGATGGCAGT |
| | TTGACCTCTCTAAAAGTCAAATGGCAAAGACCTCCTGGA |
| | AATGTGGATTCTTACAATATCACCCTGTCTCACAAAGGG |
| | ACCATCAAGGAATCCAGAGTATTAGCACCTTGGATTACT |
| | GAAACTCACTTTAAAGAGTTAGTCCCCGGTCGACTTTAT |
| | CAAGTTACTGTCAGCTGTGTCTCTGGTGAACTGTCTGCT |
| | CAGAAGATGGCAGTGGGCAGAACATTTCCAGACAAAGTT |
| | GCAAACCTGGAGGCAAACAATAATGGCAGGATGAGGTCT |
| | CTTGTAGTGAGCTGGTCGCCCCCTGCTGGAGACTGGGAG |
| | CAGTATCGGATCCTACTCTTCAATGATTCTGTGGTGCTG |
| | CTCAACATCACTGTGGGAAAGGAAGAAACACAGTATGTC |
| | ATGGATGACACGGGGCTCGTACCGGGAAGACAGTATGAG |
| | GTGGAAGTCATTGTTGAGAGTGGAAATTTGAAGAATTCT |
| | GAGCGTTGCCAAGGCAGGACAGTCCCCCTGGCTGTCCTC |
| | CAGCTTCGTGTCAAACATGCCAATGAAACCTCACTGAGT |
| | ATCATGTGGCAGACCCCTGTAGCAGAATGGGAGAAATAC |
| | ATCATTTCCCTAGCTGACAGAGACCTCTTACTGATCCAC |
| | AAGTCACTCTCCAAAGATGCCAAAGAATTCACTTTTACT |
| | GACCTGGTGCCTGGACGAAAATACATGGCTACAGTCACC |
| | AGTATTAGTGGAGACTTAAAAAATTCCTCTTCAGTAAAA |
| | GGAAGAACAGTGCCTGCCCAAGTGACTGACTTGCATGTG |
| | GCCAACCAAGGAATGACCAGTAGTCTGTTTACTAACTGG |
| | ACCCAGGCACAAGGAGACGTAGAATTTTACCAAGTCTTA |
| | CTGATCCATGAAAATGTGGTCATTAAAAATGAAAGCATC |
| | TCCAGTGAGACCAGCAGATACAGCTTCCACTCTCTCAAG |
| | TCCGGCAGCCTGTACTCCGTGGTGGTAACAACAGTGAGT |
| | GGAGGGATCTCTTCCCGACAAGTGGTTGTGGAGGGAAGA |
| | ACAGTCCCTTCCAGTGTGAGTGGAGTAACGGTGAACAAT |
| | TCCGGTCGTAATGACTACCTCAGCGTTTCCTGGCTCGTG |
| | GCGCCCGGAGATGTGGATAACTATGAGGTAACATTGTCT |
| | CATGACGGCAAGGTGGTTCAGTCCCTTGTCATTGCCAAG |
| | TCTGTCAGAGAATGTTCCTTCAGCTCCCTCACCCCAGGC |
| | CGCCTCTACACCGTGACCATAACTACAAGGAGTGGCAAG |
| | TATGAAAATCACTCCTTCAGCCAAGAGCGGACAGTGCCT |
| | GACAAAGTCCAGGGAGTCAGTGTTAGCAACTCAGCCAGG |
| | AGTGACTATTTAAGGGTATCCTGGGTGCATGCCACTGGA |
| | GACTTTGATCACTATGAAGTCACCATTAAAAACAAAAAC |
| | AACTTCATTCAAACTAAAAGCATTCCCAAGTCAGAAAAC |
| | GAATGTGTATTTGTTCAGCTAGTCCCTGGACGGTTGTAC |
| | AGTGTCACTGTTACTACAAAAAGTGGACAATATGAAGCC |
| | AATGAACAAGGGAATGGGAGAACAATTCCAGAGCCTGTT |
| | AAGGATCTAACATTGCGCAACAGGAGCACTGAGGACTTG |
| | CATGTGACTTGGTCAGGAGCTAATGGGGATGTCGACCAA |
| | TATGAGATCCAGCTGCTCTTCAATGACATGAAAGTATTT |
| | CCTCCTTTTCACCTTGTAAATACCGCAACCGAGTATCGA |
| | TTTACTTCCCTAACACCAGGCCGCCAATACAAAATTCTT |
| | GTCTTGACGATTAGCGGGGATGTACAGCAGTCAGCCTTC |
| | ATTGAGGGCTTCACAGTTCCTAGTGCTGTCAAAAATATT |
| | CACATTTCTCCCAATGGAGCAACAGATAGCCTGACGGTG |
| | AACTGGACTCCTGGTGGGGGAGACGTTGATTCCTACACG |
| | GTGTCGGCATTCAGGCACAGTCAAAAGGTTGACTCTCAG |
| | ACTATTCCCAAGCACGTCTTTGAGCACACGTTCCACAGA |
| | CTGGAGGCCGGGGAGCAGTACCAGATCATGATTGCCTCA |
| | GTCAGCGGGTCCCTGAAGAATCAGATAAATGTGGTTGGG |
| | CGGACAGTTCCAGCATCTGTCCAAGGAGTAATTGCAGAC |
| | AATGCATACAGCAGTTATTCCTTAATAGTAAGTTGGCAA |
| | AAAGCTGCTGGTGTGGCAGAAAGATATGATATCCTGCTT |
| | CTAACTGAAAATGGAATCCTTCTGCGCAACACATCAGAG |
| | CCAGCCACCACTAAGCAACACAAATTTGAAGATCTAACA |
| | CCAGGCAAGAAATACAAGATACAGATCCTAACTGTCAGT |
| | GGAGGCCTCTTTAGCAAGGAAGCCCAGACTGAAGGCCGA |
| | ACAGTCCCAGCAGCTGTCACCGACCTGAGGATCACAGAG |
| | AACTCCACCAGGCACCTGTCCTTCCGCTGGACCGCCTCA |
| | GAGGGGGAGCTCAGCTGGTACAACATCTTTTTGTACAAC |
| | CCAGATGGGAATCTCCAGGAGAGAGCTCAAGTTGACCCA |
| | CTAGTCCAGAGCTTCTCTTTCCAGAACTTGCTACAAGGC |
| | AGAATGTACAAGATGGTGATTGTAACTCACAGTGGGGAG |
| | CTGTCTAATGAGTCTTTCATATTTGGTAGAACAGTCCCA |
| | GCCTCTGTGAGTCATCTCAGGGGGTCCAATCGGAACACG |
| | ACAGACAGCCTTTGGTTCAACTGGAGTCCAGCCTCTGGG |
| | GACTTTGACTTTTATGAGCTGATTCTCTATAATCCCAAT |
| | GGCACAAAGAAGGAAAACTGGAAAGACAAGGACCTGACG |
| | GAGTGGCGGTTTCAAGGCCTTGTTCCTGGAAGGAAGTAC |
| | GTGCTGTGGGTGGTAACTCACAGTGGAGATCTCAGCAAT |
| | AAAGTCACAGCGGAGAGCAGAACAGCTCCAAGTCCTCCC |
| | AGTCTTATGTCATTTGCTGACATTGCAAACACATCCTTG |
| | GCCATCACGTGGAAAGGGCCCCCAGACTGGACAGACTAC |
| | AACGACTTTGAGCTGCAGTGGTTGCCCAGAGATGCACTT |
| | ACTGTCTTCAACCCCTACAACAACAGAAAATCAGAAGGA |
| | CGCATTGTGTATGGTCTTCGTCCAGGGAGATCCTATCAA |
| | TTCAACGTCAAGACTGTCAGTGGTGATTCCTGGAAAACT |
| | TACAGCAAACCAATTTTTGGATCTGTGAGGACAAAGCCT |
| | GACAAGATACAAAACCTGCATTGCCGGCCTCAGAACTCC |
| | ACGGCCATTGCCTGTTCTTGGATCCCTCCTGATTCTGAC |
| | TTTGATGGTTATAGTATTGAATGCCGGAAAATGGACACC |
| | CAAGAAGTTGAGTTTTCCAGAAAGCTGGAGAAAGAAAAA |
| | TCTCTGCTCAACATCATGATGCTAGTGCCCCATAAGAGG |
| | TACCTGGTGTCCATCAAAGTGCAGTCGGCCGGCATGACC |
| | AGCGAGGTGGTTGAAGACAGCACTATCACAATGATAGAC |
| | CGCCCCCCTCCTCCACCCCCACACATTCGTGTGAATGAA |
| | AAGGATGTGCTAATTAGCAAGTCTTCCATCAACTTTACT |
| | GTCAACTGCAGCTGGTTCAGCGACACCAATGGAGCTGTG |
| | AAATACTTCACAGTGGTGGTGAGAGAGGCTGATGGCAGT |
| | GATGAGCTGAAGCCAGAACAGCAGCACCCTCTCCCTTCC |
| | TACCTGGAGTACAGGCACAATGCCTCCATTCGGGTGTAT |
| | CAGACTAATTATTTTGCCAGCAAATGTGCCGAAAATCCT |
| | AACAGCAACTCCAAGAGTTTTAACATTAAGCTTGGAGCA |
| | GAGATGGAGAGCTTAGGTGGAAAACGCGATCCCACTCAG |
| | CAAAAATTCTGTGATGGACCACTGAAGCCACACACTGCC |
| | TACAGAATCAGCATTCGAGCTTTTACACAGCTCTTTGAT |
| | GAGGACCTGAAGGAATTCACAAAGCCACTCTATTCAGAC |
| | ACATTTTTTTCTTTACCCATCACTACTGAATCAGAGCCC |
| | TTGTTTGGAGCTATTGAAGGTGTGAGTGCTGGTCTGTTT |
| | TTAATTGGCATGCTAGTGGCTGTTGTTGCCTTATTGATC |
| | TGCAGACAGAAAGTGAGCCATGGTCGAGAAAGACCCTCT |
| | GCCCGTCTGAGCATTCGTAGGGATCGACCATTATCTGTC |
| | CACTTAAACCTGGGCCAGAAAGGTAACCGGAAAACTTCT |
| | TGTCCAATAAAAATAAATCAGTTTGAAGGGCATTTCATG |
| | AAGCTACAGGCTGACTCCAACTACCTTCTATCCAAGGAA |
| | TACGAGGAGTTAAAAGACGTGGGCCGAAACCAGTCATGT |
| | GACATTGCACTCTTGCCGGAGAATAGAGGGAAAAATCGA |
| | TACAACAATATATTGCCCTATGATGCCACGCGAGTGAAG |
| | CTCTCCAATGTAGATGATGATCCTTGCTCTGACTACATC |
| | AATGCCAGCTACATCCCTGGCAACAACTTCAGAAGAGAA |
| | TACATTGTCACTCAGGGACCGCTTCCTGGCACCAAGGAT |
| | GACTTCTGGAAAATGGTGTGGGAACAAAACGTTCACAAC |
| | ATCGTCATGGTGACCCAGTGTGTTGAGAAGGGCCGAGTA |
| | AAGTGTGACCATTACTGGCCAGCGGACCAGGATTCCCTC |
| | TACTATGGGGACCTCATCCTGCAGATGCTCTCAGAGTCC |
| | GTCCTGCCTGAGTGGACCATCCGGGAGTTTAAGATATGC |
| | GGTGAGGAACAGCTTGATGCACACAGACTCATCCGCCAC |
| | TTTCACTATACGGTGTGGCCAGACCATGGAGTCCCAGAA |
| | ACCACCCAGTCTCTGATCCAGTTTGTGAGAACTGTCAGG |
| | GACTACATCAACAGAAGCCCGGGTGCTGGGCCCACTGTG |
| | GTGCACTGCAGTGCTGGTGTGGGTAGGACTGGAACCTTT |
| | ATTGCATTGGACCGAATCCTCCAGCAGTTAGACTCCAAA |
| | GACTCTGTGGACATTTATGGAGCAGTGCACGACCTAAGA |
| | CTTCACAGGGTTCACATGGTCCAGACTGAGTGTCAGTAT |
| | GTCTACCTACATCAGTGTGTAAGAGATGTCCTCAGAGCA |
| | AGAAAGCTACGGAGTGAACAAGAAAACCCCTTGTTTCCA |
| | ATCTATGAAAATGTGAATCCAGAGTATCACAGAGATCCA |
| | GTCTATTCAAGGCATTGAGAATGTACCTGAAGAGCTCCT |
| | GGATAAAAATTATTCACTGTGTGATTTGTT |
| 15 | MLRHGALTALWITLSVVQTGVAEQVKCNFTLLESRVSSL |
| | SASIQWRTFASPCNFSLIYSSDTSGPMWCHPIRIDNFTY |
| | GCNPKDLQAGTVYNFRIVSLDGEESTLVLQTDPLPPARF |
| | EVNREKTASTTLQVRWTPSSGKVSWYEVQLFDHNNQKIQ |
| | EVQVQESTTWSQYTFLNLTEGNSYKVAITAVSGEKRSFP |
| | VYINGSTVPSPVKDLGISPNPNSLLISWSRGSGNVEQYR |
| | LVLMDKGAIVQDTNVDRRDTSYAFHELTPGHLYNLTIVT |
| | MASGLQNSRWKLVRTAPMEVSNLKVTNDGRLTSLNVKWQ |
| | KPPGDVDSYSITLSHQGTIKESKTLAPPVTETQFKDLVP |
| | GRLYQVTISCISGELSAEKSAAGRTVPEKVRNLVSYNEI |
| | WMKSFTVNWTPPAGDWEHYRIVLFNESLVLLNTTVGKEE |
| | THYALDGLELIPGRQYEIEVIVESGNLRNSERCQGRTVP |
| | LAVLQLRVKHANETSLGITWRAPLGEWEKYIISLMDREL |
| | LVIHKSLSKDAKEFTFTDLMPGRNYKATVTSMSGDLKQS |
| | SSIKGRTVPAQVTDLHVNNQGMTSSLFTNWTKALGDVEF |
| | YQVLLIHENVVVKNESVSSDTSRYSFRALKPGSLYSVVV |
| | TTVSGGISSRQVVAEGRTVPSSVSGVTVNNSGRNDYLSV |
| | SWLPAPGEVDHYVVSLSHEGKVDQFLIIAKSVSECSFSS |
| | LTPGRLYNVTVTTKSGNYASHSFTEERTVPDKVQGISVS |
| | NSARSDYLKVSWVHATGDFDHYEVTIKNRESFIQTKTIP |
| | KSENECEFIELVPGRLYSVTVSTKSGQYEASEQGTGRTI |
| | PEPVKDLTLLNRSTEDLHVTWSRANGDVDQYEVQLLFND |
| | MKVFPHIHLVNTATEYKFTALTPGRHYKILVLTISGDVQ |
| | QSAFIEGLTVPSTVKNIHISANGATDRLMVTWSPGGGDV |
| | DSYVVSAFRQDEKVDSQTIPKHASEHTFHRLEAGAKYRI |
| | AIVSVSGSLRNQIDALGQTVPASVQGVVAANAYSSNSLT |
| | VSWQKALGVAERYDILLLNENGLLLSNVSEPATARQHKF |
| | EDLTPGKKYKMQILTVSGGLFSKESQAEGRTVPAAVTNL |
| | RITENSSRYLSFGWTASEGELSWYNIFLYNPDRTLQERA |
| | QVDPLVQSFSFQNLLQGRMYKMVIVTHSGELSNESFIFG |
| | RTVPAAVNHLKGSHRNTTDSLWFSWSPASGDFDFYELIL |
| | YNPNGTKKENWKEKDVTEWRFQGLVPGRKYTLYVVTHSG |
| | DLSNKVTGEGRTAPSPPSLLSFADVANTSLAITWKGPPD |
| | WTDYNDFELQWFPGDALTIFNPYSSRKSEGRIVYGLHPG |
| | RSYQFSVKTVSGDSWKTYSKPISGSVRTKPDKIQNLHCR |
| | PQNSTAIACSWIPPDSDFDGYSIECRKMDTQEIEFSRKL |
| | EKEKSLLNIMMLVPHKRYLVSIKVQSAGMTSEVVEDSTI |
| | TMIDRPPQPPPHIRVNEKDVLISKSSINFTVNCSWFSDT |
| | NGAVKYFAVVVREADSMDELKPEQQHPLPSYLEYRHNAS |
| | IRVYQTNYFASKCAESPDSSSKSFNIKLGAEMDSLGGKC |
| | DPSQQKFCDGPLKPHTAYRISIRAFTQLFDEDLKEFTKP |
| | LYSDTFFSMPITTESEPLFGVIEGVSAGLFLIGMLVALV |
| | AFFICRQKASHSRERPSARLSIRRDRPLSVHLNLGQKGN |
| | RKTSCPIKINQFEGHFMKLQADSNYLLSKEYEDLKDVGR |
| | SQSCDIALLPENRGKNRYNNILPYDASRVKLCNVDDDPC |
| | SDYINASYIPGNNFRREYIATQGPLPGTKDDFWKMAWEQ |
| | NVHNIVMVTQCVEKGRVKCDHYWPADQDPLYYGDLILQM |
| | VSESVLPEWTIREFKICSEEQLDAHRLIRHFHYTVWPDH |
| | GVPETTQSLIQFVRTVRDYINRSPGAGPTVVHCSAGVGR |
| | TGTFVALDRILQQLDSKDSVDIYGAVHDLRLHRVHMVQT |
| | ECQYVYLHQCVRDVLRAKKLRNEQENPLFPIYENVNPEY |
| | HRDAIYSRH |
| 16 | EQVKCNFTLLESRVSSLSASIQWRTFASPCNFSLIYSSD |
| | TSGPMWCHPIRIDNFTYGCNPKDLQAGTVYNFRIVSLDG |
| | EESTLVLQTDPLPPARFEVNREKTASTTLQVRWTPSSGK |
| | VSWYEVQLFDHNNQKIQEVQVQESTTWSQYTFLNLTEGN |
| | SYKVAITAVSGEKRSFPVYINGSTVPSPVKDLGISPNPN |
| | SLLISWSRGSGNVEQYRLVLMDKGAIVQDTNVDRRDTSY |
| | AFHELTPGHLYNLTIVTMASGLQNSRWKLVRTAPMEVSN |
| | LKVTNDGRLTSLNVKWQKPPGDVDSYSITLSHQGTIKES |
| | KTLAPPVTETQFKDLVPGRLYQVTISCISGELSAEKSAA |
| | GRTVPEKVRNLVSYNEIWMKSFTVNWTPPAGDWEHYRIV |
| | LFNESLVLLNTTVGKEETHYALDGLELIPGRQYEIEVIV |
| | ESGNLRNSERCQGRTVPLAVLQLRVKHANETSLGITWRA |
| | PLGEWEKYIISLMDRELLVIHKSLSKDAKEFTFTDLMPG |
| | RNYKATVTSMSGDLKQSSSIKGRTVPAQVTDLHVNNQGM |
| | TSSLFTNWTKALGDVEFYQVLLIHENVVVKNESVSSDTS |
| | RYSFRALKPGSLYSVVVTTVSGGISSRQVVAEGRTVPSS |
| | VSGVTVNNSGRNDYLSVSWLPAPGEVDHYVVSLSHEGKV |
| | DQFLIIAKSVSECSFSSLTPGRLYNVTVTTKSGNYASHS |
| | FTEERTVP |
| 17 | MLSHGAGLALWITLSLLQTGLAEPERCNFTLAESKASSH |
| | SVSIQWRILGSPCNFSLIYSSDTLGAALCPTFRIDNTTY |
| | GCNLQDLQAGTIYNFRIISLDEERTVVLQTDPLPPARFG |
| | VSKEKTTSTSLHVWWTPSSGKVTSYEVQLFDENNQKIQG |
| | VQIQESTSWNEYTFFNLTAGSKYNIAITAVSGGKRSFSV |
| | YTNGSTVPSPVKDIGISTKANSLLISWSHGSGNVERYRL |
| | MLMDKGILVHGGVVDKHATSYAFHGLTPGYLYNLTVMTE |
| | AAGLQNYRWKLVRTAPMEVSNLKVTNDGSLTSLKVKWQR |
| | PPGNVDSYNITLSHKGTIKESRVLAPWITETHFKELVPG |
| | RLYQVTVSCVSGELSAQKMAVGRTFPDKVANLEANNNGR |
| | MRSLVVSWSPPAGDWEQYRILLFNDSVVLLNITVGKEET |
| | QYVMDDTGLVPGRQYEVEVIVESGNLKNSERCQGRTVPL |
| | AVLQLRVKHANETSLSIMWQTPVAEWEKYIISLADRDLL |
| | LIHKSLSKDAKEFTFTDLVPGRKYMATVTSISGDLKNSS |
| | SVKGRTVPAQVTDLHVANQGMTSSLFTNWTQAQGDVEFY |
| | QVLLIHENVVIKNESISSETSRYSFHSLKSGSLYSVVVT |
| | TVSGGISSRQVVVEGRTVPSSVSGVTVNNSGRNDYLSVS |
| | WLLAPGDVDNYEVTLSHDGKVVQSLVIAKSVRECSFSSL |
| | TPGRLYTVTITTRSGKYENHSFSQERTVPDKVQGVSVSN |
| | SARSDYLRVSWVHATGDFDHYEVTIKNKNNFIQTKSIPK |
| | SENECVFVQLVPGRLYSVTVTTKSGQYEANEQGNGRTIP |
| | EPVKDLTLRNRSTEDLHVTWSGANGDVDQYEIQLLFNDM |
| | KVFPPFHLVNTATEYRFTSLTPGRQYKILVLTISGDVQQ |
| | SAFIEGFTVPSAVKNIHISPNGATDSLTVNWTPGGGDVD |
| | SYTVSAFRHSQKVDSQTIPKHVFEHTFHRLEAGEQYQIM |
| | IASVSGSLKNQINVVGRTVPASVQGVIADNAYSSYSLIV |
| | SWQKAAGVAERYDILLLTENGILLRNTSEPATTKQHKFE |
| | DLTPGKKYKIQILTVSGGLFSKEAQTEGRTVPAAVTDLR |
| | ITENSTRHLSFRWTASEGELSWYNIFLYNPDGNLQERAQ |
| | VDPLVQSFSFQNLLQGRMYKMVIVTHSGELSNESFIFGR |
| | TVPASVSHLRGSNRNTTDSLWFNWSPASGDFDFYELILY |
| | NPNGTKKENWKDKDLTEWRFQGLVPGRKYVLWVVTHSGD |
| | LSNKVTAESRTAPSPPSLMSFADIANTSLAITWKGPPDW |
| | TDYNDFELQWLPRDALTVFNPYNNRKSEGRIVYGLRPGR |
| | SYQFNVKTVSGDSWKTYSKPIFGSVRTKPDKIQNLHCRP |
| | QNSTAIACSWIPPDSDFDGYSIECRKMDTQEVEFSRKLE |
| | KEKSLLNIMMLVPHKRYLVSIKVQSAGMTSEVVEDSTIT |
| | MIDRPPPPPPHIRVNEKDVLISKSSINFTVNCSWFSDTN |
| | GAVKYFTVVVREADGSDELKPEQQHPLPSYLEYRHNASI |
| | RVYQTNYFASKCAENPNSNSKSFNIKLGAEMESLGGKCD |
| | PTQQKFCDGPLKPHTAYRISIRAFTQLFDEDLKEFTKPL |
| | YSDTFFSLPITTESEPLFGAIE |
| 18 | LAEPERCNFTLAESKASSHSVSIQWRILGSPCNFSLIYS |
| | SDTLGAALCPTFRIDNTTYGCNLQDLQAGTIYNFRIISL |
| | DEERTVVLQTD |

HPTPβ is a member of the receptor-like family of the protein tyrosine phosphatases (PTPases). The mouse orthologue of HPTPβ is referred to as vascular endothelial protein tyrosine phosphatase (VE-PTP which has the amino acid sequence of SEQ ID NO. 15). HPTPβ is a transmembrane protein found primarily in endothelial cells that displays structural and functional similarity to cell adhesion molecules (CAMs). HPTPβ contains a single catalytic domain. One of the main functions of HPTPβ is to regulate Tie-2 receptor negatively. A HPTPβ suppressor, for example, an antibody that binds HPTPβ, can activate Tie-2 downstream signaling by inhibiting HPTPβ. Inhibition of HPTPβ by the suppressor can provide vascular stability in subjects with ocular disorders described herein. HPTPβ suppressors of the present disclosure can include antibodies, dominant-negative proteins, darpins (a genetically engineered antibody mimetic protein), peptides, aptamers (an oligonucleic acid or peptide molecule that can bind to a specific target molecule), adnectins (an antibody mimic), peptibodies (a molecule comprising an antibody Fc domain attached to at least one peptide), proteins, and nucleic acids that can bind to the extracellular domain of HPTPβ (which has the amino acid sequence of SEQ ID NO. 17) and/or inhibit at least one phosphatase activity of HPTPβ.

HPTPβ suppressors of the disclosure can include antibodies and/or antigen-binding fragments thereof that can bind to HPTPβ. The binding agent can be a monoclonal antibody. The suppressor can be a fragment of an antibody, for example, a fragment comprising one or both of heavy and light chain variable regions, F(ab′)₂, a dimer or trimer of a Fab, Fv, scFv, or a dia-, tria-, or tetrabody derived from the antibody. A suppressor can be, for example, an antibody or antigen-binding fragment that binds to HPTPβ (SEQ ID NO. 13), an antibody or antibody or antigen-binding fragment that binds to the extracellular domain of HPTPβ (SEQ ID NO. 17), an antibody or antigen-binding fragment that binds to a FN3 repeat of HPTPβ, or an antibody or antigen-binding fragment that binds to the first FN3 repeat of HPTPβ (SEQ ID NO. 18).

A HPTPβ suppressor of the disclosure can comprise the monoclonal antibody R15E6, which is immunoreactive to the extracellular domain of HPTPβ (SEQ ID NO. 17), is immunoreactive to the first FN3 repeat of HPTPβ (SEQ ID NO. 18), and can be produced by hybridoma cell line ATCC No. PTA-7580. The HPTPβ suppressor can comprise an antibody having the same or substantially the same biological characteristics of R15E6, an antibody fragment of R15E6 wherein the fragment comprises one or both of the heavy and light chain variable regions, a F(ab′)2 of R15E6, dimers or trimers of a Fab, Fv, scFv, and dia-, tria-, or tetrabodies derived from R15E6.

A HPTPβ suppressor of the disclosure can include an antibody, or an antibody fragment, variant, or derivative thereof, either alone or in combination with other amino acid sequences. The suppressor can undergo modifications, for example, enzymatic cleavage, and posttranslational modifications.

A HPTPβ suppressor of the disclosure can comprise a dominant-negative isoform of HPTPβ. In some embodiments, this dominant-negative isoform can correspond to a form of HPTPβ deficient in phosphatase activity that can compete with endogenous HPTPβ. Functional assessment of dominant-negative HPTPβ can occur via delivery of the transgene and determination of the effect on Tie2 phosphorylation.

A HPTPβ suppressor of the disclosure can comprise a plurality of HPTPβ binding sites. In some embodiments, a HPTPβ suppressor can bind to two HPTPβ molecules simultaneously, thereby bringing the two HPTPβ molecules into close proximity. A HPTPβ suppressor can bind to three HPTPβ molecules simultaneously, thereby bringing the three HPTPβ molecules into close proximity.

A HPTPβ suppressor of the disclosure can comprise a binding agent that causes the endocytosis of HPTPβ. A HPTPβ suppressor of the disclosure can comprise a binding agent that causes the degradation of HPTPβ. A HPTPβ suppressor of the disclosure can comprise a binding agent that reduces the stability of HPTPβ . A HPTPβ suppress of the disclosure can reduce the abundance of a mRNA encoding HPTPβ or the HPTPβ protein. A HPTPβ suppressor of the disclosure can generate mRNA encoding HPTPβ with altered transcript splicing. A HPTPβ suppressor of the disclosure can inhibit a post-translational modification of HPTPβ.

A HPTPβ suppressor of the disclosure can be covalently or non-covalently conjugated to another moiety. A moiety can, for example, inhibit degradation, increase half-life, increase absorption, reduce toxicity, reduce immunogenicity, and/or increase biological activity of the suppressor. Non-limiting examples of the moiety include Fc domains of immunoglobulins, polymers such as polyethylene glycol (PEG), polylysine, and dextran, lipids, cholesterol groups such as steroids, carbohydrates, dendrimers, oligosaccharides, and peptides.

### Antibody

An example of an antibody is a protein having two identical copies of a heavy chain (H) polypeptide and two identical copies of a light chain (L) polypeptide. Each of the heavy chains comprises one N-terminal variable (V_{H}) region and three C-terminal constant (C_{H}1, C_{H}2 and C_{H}3) regions. Each of the light chains comprises one N-terminal variable (V_{L}) region and one C-terminal constant (C_{L}) region. The light chain variable region is aligned with the heavy chain variable region and the light chain constant region is aligned with heavy chain constant region C_{H1}. The pairing of a heavy chain variable region and light chain variable region together forms a single antigen-binding site. Each light chain is linked to a heavy chain by one covalent disulfide bond. The two heavy chains are linked to each other by one or more disulfide bonds depending on the heavy chain isotype. Each heavy and light chain also comprises regularly-spaced intrachain disulfide bridges.

The light chain from any vertebrate species can be designated kappa or lambda based on the amino acid sequences of the constant region. Depending on the amino acid sequence of the constant region of the heavy chains, immunoglobulins can be assigned to one of five classes of immunoglobulins including IgA, IgD, IgE, IgG, and IgM, having heavy chains designated alpha, delta, epsilon, gamma and mu, respectively. The alpha and gamma classes are further divided into subclasses on the basis of differences in the sequence and function of the heavy chain constant region. Subclasses of IgA and IgG expressed by humans include IgG1, IgG2, IgG3, IgG4, IgA1 and IgA2.

A variable (V) region comprises segments that can differ extensively in sequence among antibodies. The variable region mediates antigen-binding and defines specificity of a particular antibody for its antigen. However, the variability is not evenly distributed across the span of the variable regions. Instead, the variable regions consist of relatively invariant stretches called framework regions (FR) of 15-30 amino acids separated by shorter regions of extreme variability called hypervariable regions that are each 9-12 amino acids long. The variable regions of native heavy and light chains each comprise four framework regions, largely adopting a 13-sheet configuration, connected by three hypervariable regions, which form loops connecting, and in some cases forming a part of, the 13-sheet structure. The hypervariable regions in each chain are held together in close proximity by the framework regions and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

A hypervariable region can comprise amino acid residues from a complementarity determining region (CDR), for example, around about residues 24-34 (L1), 50-56 (L2) and 89-97 (L3) in the light chain variable region, and around about 1-35 (H1), 50-65 (H2) and 95-102 (H3) in the heavy chain variable region, and/or residues from a hypervariable loop.

A monoclonal antibody can be obtained from a population of substantially homogeneous antibodies, i.e., the individual antibodies comprising the population are identical except for possible naturally occurring mutations that can be present in minor amounts. In contrast to polyclonal antibody preparations, which include different antibodies directed against different epitopes, each monoclonal antibody is directed against a single epitope, i.e., a single antigenic determinant. In addition to the specificity, the monoclonal antibodies are advantageous in that each can be synthesized uncontaminated by other antibodies.

The monoclonal antibodies used herein can be, for example, chimeric antibodies wherein a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as antigen-binding fragments of such antibodies.

An antibody fragment can comprise a portion of a multimeric antibody, for example, the antigen-binding or variable region of the intact antibody. Non-limiting examples of antibody fragments include Fab, Fab′, F(ab′)₂, dimers and trimers of Fab conjugates, Fv, scFv, minibodies, dia-, tria- and tetrabodies, and linear antibodies.

An antibody from a non-human host, such as a mouse, can be humanized by altering the amino acid sequence to be more human-like, i.e., more similar to human germline variable sequences. An example of a humanized antibody is a modified chimeric antibody. A chimeric antibody is generated as described above. The chimeric antibody is further mutated outside of the CDRs to substitute non-human sequences in the variable regions with the homologous human sequences. Another example of a humanized antibody is a CDR-grafted antibody, in which non-human CDR sequences are introduced into the human heavy and light chain variable sequences of a human antibody scaffold to replace the corresponding human CDR sequences.

A human antibody can be produced in mammalian cells, bioreactors, or transgenic animals, such as mice, chicken, sheep, goat, pig and marmoset. The transgenic animal can have a substantial portion of the human antibody-producing genome inserted into the animal's genome. The mammalian cell, bioreactor, or transgenic animal's endogenous antibody production can be rendered deficient in the production of antibodies.

A fully human monoclonal antibody corresponds to an antibody whose antigen-binding residues are fully derived from the human immunoglobulin sequence or fragments thereof undergoing selection. In some embodiments, this selection occurs using phage display techniques, in which a series of variable antibody domain is expressed on a filamentous phage coat protein and enriched for binding to a target antigen. In some embodiments, this selection occurs using transgenic animals, for example mice, rats, or rabbits, in which the entire set of endogenous immunoglobulin genes has been replaced with the entire set of human immunoglobulin genes. In some embodiments, the entire set of human immunoglobulin genes is introduced and the animal's endogenous antibody production is rendered deficient in the production of antibodies.

Non-limiting examples of epitopes include amino acids, sugars, lipids, phosphoryl, and sulfonyl groups. An epitope can have specific three dimensional structural characteristics, and/or specific charge characteristics. Epitopes can be conformational or linear.

### Identification of HPTPβ Suppressors

Suitable HPTPβ suppressors can be identified using a variety of techniques. For example, candidate agents can be screened for binding to HPTPβ. Agents that bind to HPTPβ can be screened for activity, for example, inhibition of HPTPβ-mediated dephosphorylation of Tie2. In some embodiments, the candidate agents are first screened in vivo for activity. Suitable HPTPβ suppressors can be screened for the ability to suppress steady-state levels of HPTPβ mRNA and/or protein, or for activity, for example, inhibition of HPTPβ-mediated dephosphorylation of Tie2.

### Determination of Binding Activity

The selection of a suitable assay for use in identification of a specific suppressor depends on the nature of the candidate agent to be screened. For example, where the candidates are antibodies or peptibodies, which comprise an Fc moeity, fluorescence-activated cell sorting (FACS) analysis allows the candidate agent to be selected based on the ability to bind to a cell that expresses HPTPβ. The cell can endogenously express HPTPβ or can be genetically engineered to express HPTPβ. For other candidate agents such as aptamers, other techniques can be utilized. For example, aptamers that specifically bind to HPTPβ can be selected using systematic evolution of ligands by exponential enrichment (SELEX), which selects specific aptamers through repeated rounds of in vitro selection.

### Determination of Inhibitor Activity

HPTPβ suppressors can be screened for HPTPβ mediated activity, for example, inhibition of Tie2 dephosporylation. In one suitable assay based on western blotting, human umbilical vein endothelial cells (HUVEC) are cultured in serum free media in the presence or absence of various concentrations of candidate agent, and lysates of the cells are prepared, immunoprecipitated with a Tie2 antibody, resolved by polyacrylamide gel electrophoresis, and transferred to a polyvinylidene difluoride (PVDF) membrane. Membrane-bound immunoprecipitated proteins are then serially western blotted with an antiphosphotyrosine antibody to quantify Tie2 phosphorylation followed by a Tie2 antibody to quantify total Tie2. Tie2 phosphorylation is expressed as the ratio of the anti-phosphotyrosine signal over the total Tie2 signal. Greater levels of the anti-phosphotyrosine signal indicate greater HPTPβ inhibition by the candidate agent.

### Gene Therapy

The compositions and methods of the disclosure provide for the administration of a pharmaceutical composition comprising a nucleic acid encoding a HPTPβ suppressor to a subject in need thereof, for the treatment of ocular disorders that are characterized by, for example, vascular instability, vascular leakage, and neovascularization.

### Nucleic Acid Delivery Methods

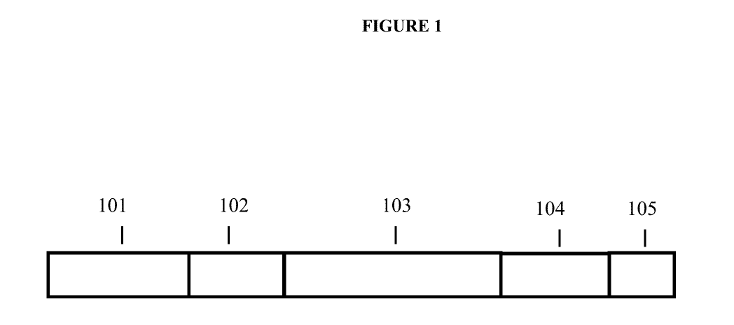

The present disclosure provides a nucleic acid encoding a HPTPβ suppressor, such as an antibody binding the HPTPβ extracellular domain (SEQ ID NO. 17), delivered by a suitable method, for example, a recombinant viral vector, to a subject in need thereof. FIG. 1 depicts a schematic representation of an illustrative therapeutic nucleic acid of the disclosure. The nucleic acid can comprise, for example, exons 103 encoding a HPTPβ suppressor, an intron 104, an enhancer region 101, a promoter region 102, and a transcription terminator region 105.

Delivery of a nucleic acid to a cell, referred to as transfection, can be accomplished by a number of methods. Viral nucleic acid delivery methods use recombinant viruses for nucleic acid transfer. Non-viral nucleic acid delivery can comprise injecting naked DNA or RNA, use of carriers including lipid carriers, polymer carriers, chemical carriers and biological carriers such as biologic membranes, bacteria, and virus-like particles, and physical/mechanical approaches. A combination of viral and non-viral nucleic acid delivery methods can be used for efficient gene therapy.

Non-viral nucleic acid transfer can include injection of naked nucleic acid, for example, nucleic acid that is not protected and/or devoid of a carrier. In vivo, naked nucleic acid can be subject to rapid degradation, low transfection levels, and poor tissue-targeting ability. Hydrodynamic injection methods can increase the targeting ability of naked nucleic acids.

Non-viral nucleic acid delivery systems can include chemical carriers. These systems can include lipoplexes, polyplexes, dendrimers, and inorganic nanoparticles. A lipoplex is a complex of a lipid and a nucleic-acid that protects the nucleic acid from degradation and facilitates entry into cells. Lipoplexes can be prepared from neutral, anionic, and/or cationic lipids. Preparation of lipoplexes with cationic lipids can facilitate encapsulation of negatively charged nucleic acids. Lipoplexes with a net positive charge can interact more efficiently with a negatively charged cell membrane. Preparation of lipoplexes with a slight excess of positive charges can confer higher transfection efficiency. Lipoplexes can enter cells by endocytosis. Once inside the cell, lipoplexes can release the nucleic acid contents into the cytoplasm. A polyplex is a complex of a polymer and a nucleic acid. Most polyplexes are prepared from cationic polymers that facilitate assembly by ionic interactions between nucleic acids and polymers. Uptake of polyplexes into cells can occur by endocytosis. Inside the cells, polyplexes require co-transfected endosomal rupture agents such as inactivated adenovirus, for the release of the polyplex particle from the endocytic vesicle. Examples of polymeric carriers include polyethyleneimine, chitosan, poly(beta-amino esters) and polyphosphoramidate. Polyplexes show low toxicity, high loading capacity, and ease of fabrication. A dendrimer is a highly branched molecule. Dendrimers can be constructed to have a positively-charged surface and/or carry functional groups that aid temporary association of the dendrimer with nucleic acids. These dendrimer-nucleic acid complexes can be used for gene therapy. The dendrimer-nucleic acid complex can enter the cell by endocytosis. Nanoparticles prepared from inorganic material can be used for nucleic acid delivery. Examples of inorganic material can include gold, silica/silicate, silver, iron oxide, and calcium phosphate. Inorganic nanoparticles with a size of less than 100 nm can be used to encapsulate nucleic acids efficiently. The nanoparticles can be taken up by the cell via endocytosis. Inside the cell, the nucleic acid can be released from the endosome without degradation. Nanoparticles based on quantum dots can be prepared and offers the use of a stable fluorescence marker coupled with gene therapy. Organically modified silica or silicate can be used to target nucleic acids to specific cells in an organism.

Non-viral nucleic acid delivery systems can include biological methods including bactofection, biological liposomes, and virus-like particles (VLPs). Bactofection method comprises using attenuated bacteria to deliver nucleic acids to a cell. Biological liposomes, such as erythrocyte ghosts and secretion exosomes, are derived from the subject receiving gene therapy to avoid an immune response. Virus-like particles (VLP) or empty viral particles are produced by transfecting cells with only the structural genes of a virus and harvesting the empty particles. The empty particles are loaded with nucleic acids to be transfected for gene therapy.

Delivery of nucleic acids can be enhanced by physical methods. Examples of physical methods include electroporation, gene gun, sonoporation, and magnetofection. The electroporation method uses short high-voltage pulses to transfer nucleic acid across the cell membrane. These pulses can lead to formation of temporary pores in the cell membrane, thereby allowing nucleic acid to enter the cell. Electroporation can be efficient for a broad range of cells. Electron-avalanche transfection is a type of electroporation method that uses very short, for example, microsecond, pulses of high-voltage plasma discharge for increasing efficiency of nucleic acid delivery. The gene gun method utilizes nucleic acid-coated gold particles that are shot into the cell using high-pressure gas. Force generated by the gene gun allows penetration of nucleic acid into the cells, while the gold is left behind on a stopping disk. The sonoporation method uses ultrasonic frequencies to modify permeability of cell membrane. Change in permeability allows uptake of nucleic acid into cells. The magnetofection method uses a magnetic field to enhance nucleic acid uptake. In this method, nucleic acid is complexed with magnetic particles. A magnetic field is used to concentrate the nucleic acid complex and bring them in contact with cells.

Viral nucleic acid delivery systems use recombinant viruses to deliver nucleic acids for gene therapy. Non-limiting examples of viruses that can be used to deliver nucleic acids include retrovirus, adenovirus, herpes simplex virus, adeno-associated virus, vesicular stomatitis virus, reovirus, vaccinia, pox virus, and measles virus.

Retroviral vectors can be used in the disclosure. Retrovirus is an enveloped virus that contains a single-stranded RNA genome. Retroviruses can integrate inside a host cell via reverse transcription. Retroviruses can enter a host cell by binding to specific membrane-bound receptors. Inside the host cell cytoplasm, retroviral reverse transcriptase generates double-stranded DNA from the viral RNA genome template. Retroviral enzyme integrase incorporates the new viral DNA into host cell genome, where the viral DNA is transcribed and translated along with host cell genes. Retroviral gene therapy vectors can be used for chromosomal integration of the transferred vector genomes, thereby leading to stable genetic modification of treated cells. Non-limiting examples of retroviral vectors include Moloney murine leukemia viral (MMLV) vectors, HIV-based viral vectors, gammaretroviral vectors, C-type retroviral vectors, and lentiviral vectors. Lentivirus is a subclass of retrovirus. While some retroviruses can infect only dividing cells, lentiviruses can infect and integrate into the genome of actively dividing cells and non-dividing cells.

Adenovirus-based vectors can be used in the disclosure. Adenovirus is a non-enveloped virus with a linear double-stranded genome. Adenoviruses can enter host cells using interactions between viral surface proteins and host cell receptors that lead to endocytosis of the adenovirus particle. Once inside the host cell cytoplasm, the adenovirus particle is released by the degradation of the endosome. Using cellular microtubules, the adenovirus particle gains entry into the host cell nucleus, where adenoviral DNA is released. Inside the host cell nucleus, the adenoviral DNA is transcribed and translated. Adenoviral DNA is not integrated into the host cell genome. Adenoviral DNA is not replicated during host cell division. Gene therapy using adenoviral vectors can require multiple administrations if the host cell population is replicating.

Herpes simplex virus (HSV)-based vectors can be used in the disclosure. HSV is an enveloped virus with a linear double-stranded DNA genome. Interactions between surface proteins on the host cell and HSV lead to pore formation in the host cell membrane. These pores allow HSV to enter the host cell cytoplasm. Inside the host cell, HSV uses the nuclear entry pore to enter the host cell nucleus where HSV DNA is released. HSV can persist in host cells in a state of latency. Herpes simplex virus 1 and 2 (HSV-1 and HSV-2), also known as human herpes virus 1 and 2 (HHV-1 and HHV-2), are members of the herpes virus family.

Alphavirus-based vectors can be used to deliver nucleic acids. Examples of alphavirus-based vectors include vectors derived from semliki forest virus and sindbis virus. Alphavirus-based vectors can provide high transgene expression and the ability to transduce a wide variety of cells. Alphavirus vectors can be modified to target specific tissues. Alphaviruses can persist in a latent state in host cells, thereby offering the advantage of long-term nucleic acid expression in the cell.

Pox/vaccinia-based vectors such as orthopox or avipox vectors can be used in the disclosure. Pox virus is a double stranded DNA virus that can infect diving and non-dividing cells. Pox viral genome can accommodate up to 25 kb transgenic sequence. Multiple genes can be delivered using a single vaccinia viral vector.

In one aspect, the present disclosure provides a recombinant virus, such as an adeno-associated virus (AAV), as a vector to deliver a nucleic acid encoding a HPTPβ suppressor to a subject in need thereof.

Adeno-associated virus (AAV) is a small, nonenveloped virus that belongs to the Parvoviridae family. AAV genome is a linear single-stranded DNA molecule of about 4,800 nucleotides. The AAV DNA comprises two inverted terminal repeats (ITRs) at both ends of the genome and two sets of open reading frames. The ITRs serve as origins of replication for the viral DNA and as integration elements. The open reading frames encode for the Rep (non-structural replication) and Cap (structural capsid) proteins. AAV can infect dividing cells and quiescent cells. AAV is common in the general population and can persist naturally in the host.

AAV can be engineered for use as a gene therapy vector by substituting the coding sequence for both AAV genes with a transgene (transferred nucleic acid) to be delivered to a cell. The subsitution eliminates immunologic or toxic side effects due to expression of viral genes. The transgene can be placed between the two ITRs (145 bp) on the AAV DNA molecule. AAV-based vectors can transencapsidate the genome allowing large variations in vector biology and tropism.

When producing recombinant AAV (rAAV), the viral genes and/or adenovirus genes providing helper functions to AAV can be supplied in trans to allow for production of the rAAV particles. In this way, rAAV can be produced through a three-plasmid system, decreasing the probability of production of wild-type virus.

AAV vector of the present disclosure can be generated using any AAV serotype. Non-limiting examples of serotypes include AAV1, AAV2, AAV2.5, AAV3, AAV4, AAV5, AAV6, AAV7, AAV8, AAV9, AAV10, AAV11, AAV12, rh10, and hybrids thereof.

AAV vectors can be modified for immune evasion or to enhance therapeutic output. The modifications can include genetic manipulation of the viral capsid. Proteins in the viral capsid can be rationally designed. The viral capsid can be modified by introducing exogenous agents such as antibodies, copolymers, and cationic lipids to evade the immune system. AAV vectors can be engineered to enhance the targeting ability. Targeting peptides and/or ligands can be inserted onto the capsid surface to enhance transduction into specific tissue. Capsid proteins from more than one serotype of AAV can be combined to produce a mosaic AAV vector comprising a capsid particle with enhanced targeting ability of the AAV vector. Tissue-specific promoters can be added to the viral vector to express the transgene in desired tissue types.

AAV vector can be modified to be self-complementary. A self-complementary AAV vector can comprise both strands of the viral DNA, thereby alleviating the requirement for host-cell second-strand DNA synthesis. The use of self-complementary AAV vectors can promote efficient transfer of nucleic acids into host genome.

A pseudotyped virus can be used for the delivery of nucleic acids. Psuedotyping involves substitution of endogenous envelope proteins of the virus by envelope proteins from other viruses or chimeric proteins. The foreign envelope proteins can confer a change in host tropism or alter stability of the virus. An example of a pseudotyped virus useful for gene therapy includes vesicular stomatitis virus G-pseudotyped lentivirus (VSV G-pseudotyped lentivirus) that is produced by coating the lentivirus with the envelope G-protein from Vesicular stomatitis virus. VSV G-pseudotyped lentivirus can transduce almost all mammalian cell types.

A hybrid vector having properties of two or more vectors can be used for nucleic acid delivery to a host cell. Hybrid vectors can be engineered to reduce toxicity or improve therapeutic transgene expression in target cells. Non-limiting examples of hybrid vectors include AAV/adenovirus hybrid vectors, AAV/phage hybrid vectors, and retrovirus/adenovirus hybrid vectors.

A viral vector can be replication-competent. A replication-competent vector contains all the genes necessary for replication, making the genome lengthier than replication-defective viral vectors. A viral vector can be replication-defective, wherein the coding region for the genes essential for replication and packaging are deleted or replaced with other genes. Replication-defective viruses can transduce host cells and transfer the genetic material, but do not replicate. A helper virus can be supplied to help a replication-defective virus replicate.

A viral vector can be derived from any source, for example, humans, non-human primates, dogs, fowl, mouse, cat, sheep, and pig.

The composition and methods of the disclosure provide for the delivery of a nucleic acid that encodes for a HPTPβ suppressor to a subject in need thereof. The nucleic acid can be delivered by a viral vector, for example, an adeno-associated virus (AAV), adenovirus, retrovirus, herpes simplex virus, lentivirus, poxvirus, hemagglutinating virus of Japan-liposome (HVJ) complex, Moloney murine leukemia virus, or HIV-based virus. The nucleic acid can be delivered by a suitable non-viral method, for example, injection of naked nucleic acid, use of carriers such as lipid, polymer, biological or chemical carriers, or physical/mechanical approaches. The nucleic acid can be delivered by a combination of viral and non-viral methods.

The nucleic acid of the disclosure can be generated using any method. The nucleic acid can be synthetic, recombinant, isolated, and/or purified. The nucleic acid can comprise, for example, a nucleic acid sequence that encodes antibody R15E6 produced by hybridoma cell line ATCC No. PTA-7580.

A vector of the disclosure can comprise one or more nucleic acid sequences, each of which encodes one or more of the heavy and/or light chain polypeptides of a HPTPβ-binding antibody. In one embodiment, the vector can comprise a single nucleic acid sequence that encodes the two heavy chain polypeptides and the two light chain polypeptides of the HPTPβ-binding antibody. In another embodiment, the vector can comprise a first nucleic acid sequence that encodes both heavy chain polypeptides of HPTPβ antibody, and a second nucleic acid sequence that encodes both light chain polypeptides of HPTPβ antibody. In some embodiments, the vector can comprise a first nucleic acid sequence encoding a first heavy chain polypeptide of HPTPβ, a second nucleic acid sequence encoding a second heavy chain polypeptide of HPTPβ, a third nucleic acid sequence encoding a first light chain polypeptide of HPTPβ, and a fourth nucleic acid sequence encoding a second light chain polypeptide of HPTPβ.

A vector of the present disclosure can comprise one or more types of nucleic acids. The nucleic acids can include DNA or RNA. RNA nucleic acids can include a transcript of a gene of interest, for example, a HPTPβ suppressor, introns, untranslated regions, and termination sequences, or short interfering RNAs targeting HPTPβ. DNA nucleic acids can include the gene of interest, promoter sequences, untranslated regions, and termination sequences. A combination of DNA and RNA can be used. The nucleic acids can be double-stranded or single-stranded. The nucleic acid can include non-natural or altered nucleotides.

A vector of the disclosure can comprise additional nucleic acid sequences including promoters, enhancers, repressors, insulators, polyadenylation signals (polyA), untranslated regions (UTRs), termination sequences, transcription terminators, internal ribosome entry sites (IRES), introns, origins of replication sequence, primer binding sites, att sites, encapsidation sites, polypurine tracts, Long Terminal Repeats (LTRs), and linker sequences. The vector can be modified to target specific cells, for example, cancer cells, or to a tissue, for example, retina.

Expression of a suppressor of HPTPβ can be under the control of a regulatory sequence. The regulatory sequence can comprise a promoter. Promoters from any suitable source including virus, mammal, human, insect, plant, yeast, and bacteria, can be used. Tissue-specific promoters can be used. Promoters can be constitutive, inducible, or repressible. Promoters can be unidirectional (initiating transcription in one direction) or bi-directional (initiating transcription in either a 3′ or 5′ direction). Non-limiting examples of promoters include the T7 bacterial expression system, pBAD (araA) bacterial expression system, the cytomegalovirus (CMV) promoter, the SV40 promoter, the Rous sarcoma virus promoter, MMT promoter, EF-1 alpha promoter, UB6 promoter, chicken beta-actin promoter, CAG promoter, RPE65 promoter, opsin promoter, HIV-1 promoter, HIV-2 promoter, AAV promoter, adenovirus promoters such as from the E1A, E2A, or MLP region, cauliflower mosaic virus promoter, HSV-TK promoter, avian sarcoma virus promoter, MLV promoter, MMTV promoter, and rat insulin promoter. Inducible promoters can include, for example, the Tet system, the ecdysone inducible system, the T-REX™ system, LACSWITCH™ System, and the Cre-ERT tamoxifen inducible recombinase system.

Promoter sequences or any associated regulatory sequences can comprise any number of nucleotides. Promoter sequences or any associated regulatory sequences can comprise, for example, at least 150 bases or base pairs, at least 200 bases or base pairs, at least 300 bases or base pairs, at least 400 bases or base pairs, at least 500 bases or base pairs, at least 600 bases or base pairs, at least 700 bases or base pairs, at least 800 bases or base pairs, at least 900 bases or base pairs, at least 1000 bases or base pairs, at least 1500 bases or base pairs, at least 2000 bases or base pairs, at least 3000 bases or base pairs, at least 4000 bases or base pairs, at least 5000 bases or base pairs, or at least 10000 bases or base pairs. Promoter sequences and any associated regulatory sequences can comprise, for example, at most 150 bases or base pairs, at most 200 bases or base pairs, at most 300 bases or base pairs, at most 400 bases or base pairs, at most 500 bases or base pairs, at most 600 bases or base pairs, at most 700 bases or base pairs, at most 800 bases or base pairs, at most 900 bases or base pairs, at most 1000 bases or base pairs, at most 1500 bases or base pairs, at most 2000 bases or base pairs, at most 3000 bases or base pairs, at most 4000 bases or base pairs, at most 5000 bases or base pairs, or at most 10000 bases or base pairs.

An intron sequence can comprise any number of nucleotides. An intron can comprise, for example, at least 1 base or base pairs, at least 50 bases or base pairs, at least 100 bases or base pairs, at least 150 bases or base pairs, at least 200 bases or base pairs, at least 300 bases or base pairs, at least 400 bases or base pairs, at least 500 bases or base pairs, at least 600 bases or base pairs, at least 700 bases or base pairs, at least 800 bases or base pairs, at least 900 bases or base pairs, at least 1000 bases or base pairs, at least 1500 bases or base pairs, at least 2000 bases or base pairs, at least 3000 bases or base pairs, at least 4000 bases or base pairs, or at least 5000 bases or base pairs. In some embodiments, an intron can comprise, for example, at 1 base or base pairs, at most 50 bases or base pairs, at most 100 bases or base pairs, at most 150 bases or base pairs, at most 200 bases or base pairs, at most 300 bases or base pairs, at most 400 bases or base pairs, at most 500 bases or base pairs, at most 600 bases or base pairs, at most 700 bases or base pairs, at most 800 bases or base pairs, at most 900 bases or base pairs, at most 1000 bases or base pairs, at most 1500 bases or base pairs, at most 2000 bases or base pairs, at most 3000 bases or base pairs, at most 4000 bases or base pairs, or at most 5000 bases or base pairs.

A polyA sequence can comprise any number of nucleotides. A polyA sequence can comprise a length of about 1 to about 10 bases or base pairs, about 10 to about 20 bases or base pairs, about 20 to about 50 bases or base pairs, about 50 to about 100 bases or base pairs, about 100 to about 500 bases or base pairs, about 500 to about 1000 bases or base pairs, about 1000 to about 2000 bases or base pairs, about 2000 to about 3000 bases or base pairs, about 3000 to about 4000 bases or base pairs, about 4000 to about 5000 bases or base pairs, about 5000 to about 6000 bases or base pairs, about 6000 to about 7000 bases or base pairs, about 7000 to about 8000 bases or base pairs, about 8000 to about 9000 bases or base pairs, or about 9000 to about 10000 bases or base pairs in length. A polyA sequence can comprise a length of for example, at least 1 base or base pair, at least 2 bases or base pairs, at least 3 bases or base pairs, at least 4 bases or base pairs, at least 5 bases or base pairs, at least 6 bases or base pairs, at least 7 bases or base pairs, at least 8 bases or base pairs, at least 9 bases or base pairs, at least 10 bases or base pairs, at least 20 bases or base pairs, at least 30 bases or base pairs, at least 40 bases or base pairs, at least 50 bases or base pairs, at least 60 bases or base pairs, at least 70 bases or base pairs, at least 80 bases or base pairs, at least 90 bases or base pairs, at least 100 bases or base pairs, at least 200 bases or base pairs, at least 300 bases or base pairs, at least 400 bases or base pairs, at least 500 bases or base pairs, at least 600 bases or base pairs, at least 700 bases or base pairs, at least 800 bases or base pairs, at least 900 bases or base pairs, at least 1000 bases or base pairs, at least 2000 bases or base pairs, at least 3000 bases or base pairs, at least 4000 bases or base pairs, at least 5000 bases or base pairs, at least 6000 bases or base pairs, at least 7000 bases or base pairs, at least 8000 bases or base pairs, at least 9000 bases or base pairs, or at least 10000 bases or base pairs in length. A polyA sequence can comprise a length of at most 1 base or base pair, at most 2 bases or base pairs, at most 3 bases or base pairs, at most 4 bases or base pairs, at most 5 bases or base pairs, at most 6 bases or base pairs, at most 7 bases or base pairs, at most 8 bases or base pairs, at most 9 bases or base pairs, at most 10 bases or base pairs, at most 20 bases or base pairs, at most 30 bases or base pairs, at most 40 bases or base pairs, at most 50 bases or base pairs, at most 60 bases or base pairs, at most 70 bases or base pairs, at most 80 bases or base pairs, at most 90 bases or base pairs, at most 100 bases or base pairs, at most 200 bases or base pairs, at most 300 bases or base pairs, at most 400 bases or base pairs, at most 500 bases or base pairs, at most 600 bases or base pairs, at most 700 bases or base pairs, at most 800 bases or base pairs, at most 900 bases or base pairs, at most 1000 bases or base pairs, at most 2000 bases or base pairs, at most 3000 bases or base pairs, at most 4000 bases or base pairs, at most 5000 bases or base pairs, at most 6000 bases or base pairs, at most 7000 bases or base pairs, at most 8000 bases or base pairs, at most 9000 bases or base pairs, or at most 10000 bases or base pairs in length.

An untranslated region can comprise any number of nucleotides. An untranslated region can comprise a length of about 1 to about 10 bases or base pairs, about 10 to about 20 bases or base pairs, about 20 to about 50 bases or base pairs, about 50 to about 100 bases or base pairs, about 100 to about 500 bases or base pairs, about 500 to about 1000 bases or base pairs, about 1000 to about 2000 bases or base pairs, about 2000 to about 3000 bases or base pairs, about 3000 to about 4000 bases or base pairs, about 4000 to about 5000 bases or base pairs, about 5000 to about 6000 bases or base pairs, about 6000 to about 7000 bases or base pairs, about 7000 to about 8000 bases or base pairs, about 8000 to about 9000 bases or base pairs, or about 9000 to about 10000 bases or base pairs in length. An untranslated region can comprise a length of for example, at least 1 base or base pair, at least 2 bases or base pairs, at least 3 bases or base pairs, at least 4 bases or base pairs, at least 5 bases or base pairs, at least 6 bases or base pairs, at least 7 bases or base pairs, at least 8 bases or base pairs, at least 9 bases or base pairs, at least 10 bases or base pairs, at least 20 bases or base pairs, at least 30 bases or base pairs, at least 40 bases or base pairs, at least 50 bases or base pairs, at least 60 bases or base pairs, at least 70 bases or base pairs, at least 80 bases or base pairs, at least 90 bases or base pairs, at least 100 bases or base pairs, at least 200 bases or base pairs, at least 300 bases or base pairs, at least 400 bases or base pairs, at least 500 bases or base pairs, at least 600 bases or base pairs, at least 700 bases or base pairs, at least 800 bases or base pairs, at least 900 bases or base pairs, at least 1000 bases or base pairs, at least 2000 bases or base pairs, at least 3000 bases or base pairs, at least 4000 bases or base pairs, at least 5000 bases or base pairs, at least 6000 bases or base pairs, at least 7000 bases or base pairs, at least 8000 bases or base pairs, at least 9000 bases or base pairs, or at least 10000 bases or base pairs in length. An untranslated region can comprise a length of at most 1 base or base pair, at most 2 bases or base pairs, at most 3 bases or base pairs, at most 4 bases or base pairs, at most 5 bases or base pairs, at most 6 bases or base pairs, at most 7 bases or base pairs, at most 8 bases or base pairs, at most 9 bases or base pairs, at most 10 bases or base pairs, at most 20 bases or base pairs, at most 30 bases or base pairs, at most 40 bases or base pairs, at most 50 bases or base pairs, at most 60 bases or base pairs, at most 70 bases or base pairs, at most 80 bases or base pairs, at most 90 bases or base pairs, at most 100 bases or base pairs, at most 200 bases or base pairs, at most 300 bases or base pairs, at most 400 bases or base pairs, at most 500 bases or base pairs, at most 600 bases or base pairs, at most 700 bases or base pairs, at most 800 bases or base pairs, at most 900 bases or base pairs, at most 1000 bases or base pairs, at most 2000 bases or base pairs, at most 3000 bases or base pairs, at most 4000 bases or base pairs, at most 5000 bases or base pairs, at most 6000 bases or base pairs, at most 7000 bases or base pairs, at most 8000 bases or base pairs, at most 9000 bases or base pairs, or at most 10000 bases or base pairs in length.

A linker sequence can comprise any number of nucleotides. A linker sequence can comprise a length of about 1 to about 10 bases or base pairs, about 10 to about 20 bases or base pairs, about 20 to about 50 bases or base pairs, about 50 to about 100 bases or base pairs, about 100 to about 500 bases or base pairs, about 500 to about 1000 bases or base pairs, about 1000 to about 2000 bases or base pairs, about 2000 to about 3000 bases or base pairs, about 3000 to about 4000 bases or base pairs, about 4000 to about 5000 bases or base pairs, about 5000 to about 6000 bases or base pairs, about 6000 to about 7000 bases or base pairs, about 7000 to about 8000 bases or base pairs, about 8000 to about 9000 bases or base pairs, or about 9000 to about 10000 bases or base pairs in length. A linker sequence can comprise a length of for example, at least 1 base or base pair, at least 2 bases or base pairs, at least 3 bases or base pairs, at least 4 bases or base pairs, at least 5 bases or base pairs, at least 6 bases or base pairs, at least 7 bases or base pairs, at least 8 bases or base pairs, at least 9 bases or base pairs, at least 10 bases or base pairs, at least 20 bases or base pairs, at least 30 bases or base pairs, at least 40 bases or base pairs, at least 50 bases or base pairs, at least 60 bases or base pairs, at least 70 bases or base pairs, at least 80 bases or base pairs, at least 90 bases or base pairs, at least 100 bases or base pairs, at least 200 bases or base pairs, at least 300 bases or base pairs, at least 400 bases or base pairs, at least 500 bases or base pairs, at least 600 bases or base pairs, at least 700 bases or base pairs, at least 800 bases or base pairs, at least 900 bases or base pairs, at least 1000 bases or base pairs, at least 2000 bases or base pairs, at least 3000 bases or base pairs, at least 4000 bases or base pairs, at least 5000 bases or base pairs, at least 6000 bases or base pairs, at least 7000 bases or base pairs, at least 8000 bases or base pairs, at least 9000 bases or base pairs, or at least 10000 bases or base pairs in length. A linker sequence can comprise a length of at most 1 base or base pair, at most 2 bases or base pairs, at most 3 bases or base pairs, at most 4 bases or base pairs, at most 5 bases or base pairs, at most 6 bases or base pairs, at most 7 bases or base pairs, at most 8 bases or base pairs, at most 9 bases or base pairs, at most 10 bases or base pairs, at most 20 bases or base pairs, at most 30 bases or base pairs, at most 40 bases or base pairs, at most 50 bases or base pairs, at most 60 bases or base pairs, at most 70 bases or base pairs, at most 80 bases or base pairs, at most 90 bases or base pairs, at most 100 bases or base pairs, at most 200 bases or base pairs, at most 300 bases or base pairs, at most 400 bases or base pairs, at most 500 bases or base pairs, at most 600 bases or base pairs, at most 700 bases or base pairs, at most 800 bases or base pairs, at most 900 bases or base pairs, at most 1000 bases or base pairs, at most 2000 bases or base pairs, at most 3000 bases or base pairs, at most 4000 bases or base pairs, at most 5000 bases or base pairs, at most 6000 bases or base pairs, at most 7000 bases or base pairs, at most 8000 bases or base pairs, at most 9000 bases or base pairs, or at most 10000 bases or base pairs in length.

A vector of the disclosure can comprise nucleic acids encoding a selectable marker. The selectable marker can be positive, negative or bifunctional. The selectable marker can be an antibiotic-resistance gene. Examples of antibiotic resistance genes include markers conferring resistance to kanamycin, gentamicin, ampicillin, chloramphenicol, tetracycline, doxycycline, hygromycin, puromycin, zeomycin, or blasticidin. The selectable marker can allow imaging of the host cells, for example, a fluorescent protein. Examples of imaging marker genes include GFP, eGFP, RFP, CFP, YFP, dsRed, Venus, mCherry, mTomato, and mOrange.

A vector of the disclosure can comprise fusion proteins. The fusion partner can comprise a signal polypeptide that targets the protein to the desired site. The fusion partner can comprise a polypeptide tag, for example, a poly-His and/or a Flag peptide, that facilitates purification of the protein. The fusion partner can comprise an imaging tag, for example, a fluorescent protein, for imaging the cells. A vector of the disclosure can comprise chemical conjugates.

A vector of the disclosure can comprise components to confer additional properties to the vector. These properties can include targeting of the vector to a specific tissue, uptake of vector into a host cell, entry of nucleic acid into nucleus, incorporation of nucleic acid into host cell genome, transgene expression in host cell, immune evasion, and vector stability.

A vector of the disclosure can be generated by any suitable methods. The method can include use of transgenic cells including for example, mammalian cells such as HEK293, insect cells such as Sf9, animal cells or fungal cells.

A viral vector of the disclosure can be measured as plaque forming units (pfu). The pfu of a viral vector can be, for example, from about 10¹ to about 10¹⁸ pfu. A viral vector of the disclosure can be, for example, at least 10¹, at least 10², at least 10³, at least 10⁴, at least 10⁵, at least 10⁶, at least 10⁷, at least 10⁸, at least 10⁹, at least 10 ¹⁰, at least 10¹¹, at least 10¹², at least 10¹³, at least 10¹⁴, at least 10¹⁵, at least 10¹⁶, at least 10¹⁷, or at least 10¹⁸ pfu. A viral vector of the disclosure can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸ pfu.

A viral vector of the disclosure can be measured as vector genomes. A viral vector of the disclosure can be, for example, from about 10¹ to about 10¹⁸ vector genomes. A viral vector of the disclosure can be, for example, at least 10¹, at least 10², at least 10³, at least 10⁴, at least 10⁵, at least 10⁶, at least 10⁷, at least 10⁸, at least 10⁹ , at least 10¹⁰, at least 10¹¹, at least 10¹², at least 10¹³, at least 10¹⁴, at least 10¹⁵, at least 10¹⁶, at least 10¹⁷, or at least 10¹⁸ vector genomes. A viral vector of the disclosure can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10 ¹⁴, at most 10¹⁵, at most 10¹⁶, at most 10¹⁷, or at most 10¹⁸ vector genomes.

A viral vector of the disclosure can be measured using multiplicity of infection (MOI). MOI can be, for example, the ratio, or multiple of vector or viral genomes to the cells to which the nucleic acid can be delivered. A viral vector of the disclosure can be, for example, from about 10¹ to about 10¹⁸ MOI. A viral vector of the disclosure can be, for example, at least about 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸ MOI. A viral vector of the disclosure can be, for example, at most 10¹, at most 10², at most 10³, at most 10⁴, at most 10⁵, at most 10⁶, at most 10⁷, at most 10⁸, at most 10⁹, at most 10¹⁰ at most 10¹¹, at most 10¹², at most 10¹³, at most 10 ¹⁴, at most 10¹⁵, at most 10¹⁶, at most 10¹⁷, or at most 10¹⁸ MOI.

Any suitable amount of nucleic acid can be used with the compositions and methods of the disclosure. The amount of nucleic acid can be, for example, from about 1 pg to about 1 ng. The amount of nucleic acid can be, for example, from about 1 ng to about 1 μg. The amount of nucleic acid can be, for example, from about 1 μg to about 1 mg. The amount of nucleic acid can be, for example, from about 1 mg to about 1 g. The amount of nucleic acid can be, for example, from about 1 g to about 5 g. The amount of nucleic acid can be, for example, at least 1 pg, at least 10 pg, at least 100 pg, at least 200 pg, at least 300 pg, at least 400 pg, at least 500 pg, at least 600 pg, at least 700 pg, at least 800 pg, at least 900 pg, at least 1 ng, at least 10 ng, at least 100 ng, at least 200 ng, at least 300 ng, at least 400 ng, at least 500 ng, at least 600 ng, at least 700 ng, at least 800 ng, at least 900 ng, at least 1 μg, at least 10 μg, at least 100 μg, at least 200 μg, at least 300 μg, at least 400 μg, at least 500 μg, at least 600 μg, at least 700 μg, at least 800 μg, at least 900 μg, at least 1 mg, at least 10 mg, at least 100 mg, at least 200 mg, at least 300 mg, at least 400 mg, at least 500 mg, at least 600 mg, at least 700 mg, at least 800 mg, at least 900 mg, at least 1 g, at least 2 g, at least 3 g, at least 4 g, or at least 5 g. The amount of nucleic acid can be, for example, at most 1 pg, at most 10 pg, at most 100 pg, at most 200 pg, at most 300 pg, at most 400 pg, at most 500 pg, at most 600 pg, at most 700 pg, at most 800 pg, at most 900 pg, at most 1 ng, at most 10 ng, at most 100 ng, at most 200 ng, at most 300 ng, at most 400 ng, at most 500 ng, at most 600 ng, at most 700 ng, at most 800 ng, at most 900 ng, at most 1 μg, at most 10 μg, at most 100 μg, at most 200 μg, at most 300 μg, at most 400 μg, at most 500 μg, at most 600 μg, at most 700 μg, at most 800 μg, at most 900 μg, at most 1 mg, at most 10 mg, at most 100 mg, at most 200 mg, at most 300 mg, at most 400 mg, at most 500 mg, at most 600 mg, at most 700 mg, at most 800 mg, at most 900 mg, at most 1 g, at most 2 g, at most 3 g, at most 4 g, or at most 5 g.

A viral vector of the disclosure can be measured as recombinant viral particles. A viral vector of the disclosure can be, for example, from about 10¹ to about 10¹⁸ recombinant viral particles. A viral vector of the disclosure can be, for example, at least about 10¹, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, at least about 10¹⁶, at least about 10¹⁷, or at least about 10¹⁸ recombinant viral particles. A viral vector of the disclosure can be, for example, at most about 10¹, at most about 10², at most about 10³, at most about 10⁴, at most about 10⁵, at most about 10⁶, at most about 10⁷, at most about 10⁸, at most about 10⁹, at most about 10¹⁰, at most about 10¹¹, at most about 10¹², at most about 10¹³, at most about 10 ¹⁴, at most about 10¹⁵, at most about 10¹⁶, at most about 10¹⁷, or at most about 10¹⁸ recombinant viral particles.

A RNA interference (RNAi) system can be used to modify a target of the disclosure. RNAi is a targeted mRNA degradation system comprising an endogenous nuclease that is guided by specific short RNA molecules to recognize and cleave specific mRNA sequences, for example, a target mRNA in a subject. The RNAi system can be used in conjunction with other nucleic acid delivery methods such as viral vectors and non-viral methods as described herein. A zinc finger nuclease (ZFN) system can be used to modify a target or deliver a nucleic acid of the disclosure. The ZFN system is a targeted genome-editing system comprising a zinc finger nuclease that is engineered to recognize and cleave specific DNA sequences, for example, a genomic locus in a subject. The ZFN can modify the genomic locus, for example, by cleaving the genomic locus, thus generating mutations that result in loss of function of the target sequence. The ZFN can also modify the genomic locus, for example, by cleaving the genomic locus, and adding a transgene, for example, a therapeutic nucleic acid of the disclosure. The ZFN system can be used in conjunction with other nucleic acid delivery methods such as viral vectors and non-viral methods as described herein.

A transcription activator-like effector nuclease (TALEN) system can be used to modify a target or deliver a nucleic acid of the disclosure, The TALEN system is a targeted genome-editing system comprising transcription activator-like effectors that are engineered to recognize and cleave specific DNA sequences, for example, a genomic locus in a subject. The TALEN can modify the genomic locus, for example, by cleaving the genomic locus, thus generating mutations that result in loss of function of the target sequence. The TALEN can also modify the genomic locus, for example, by cleaving the genomic locus, and adding a transgene, for example, a therapeutic nucleic acid of the disclosure. The TALEN system can be used in conjunction with other nucleic acid delivery methods such as viral vectors and non-viral methods as described herein.

A Clustered Regularly Interspaced Short Palindromic Repeats (CRISPR)-CRISPR associated (Cas) (CRISPR-Cas) system can be used to modify a target or deliver a nucleic acid of the disclosure. The CRIPSR-Cas system is a targeted genome-editing system comprising a Cas nuclease that is guided to specific DNA sequences, for example, a genomic locus in a subject, by a guide RNA molecule. The Cas nuclease can modify the genomic locus, for example, by cleaving the genomic locus, thus generating mutations that result in loss of function of the target sequence. The Cas nuclease can also modify the genomic locus, for example, by cleaving the genomic locus, and adding a transgene, for example, a therapeutic nucleic acid of the disclosure. The CRIPSR/Cas system can be used in conjunction with other nucleic acid delivery methods such as viral vectors and non-viral methods as described herein.

A CRISPR interference (CRISPRi) system can be used to modify the expression of a target of the disclosure. The CRISPRi system is a targeted gene regulatory system comprising a nuclease deficient Cas enzyme fused to a transcriptional regulatory domain that is guided to specific DNA sequences, for example, a genomic locus in a subject, by a guide RNA molecule. The Cas/regulator fusion protein can occupy the genomic locus and induce, for example, transcriptional repression of the target gene through the function of a negative regulatory domain fused to the Cas protein. The CRISPRi system can be used in conjunction with other nucleic acid delivery methods such as viral vectors and non-viral methods as described herein.

### Pharmaceutical Compositions

A pharmaceutical composition of the invention can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, intramuscular, oral, parenteral, ophthalmic, and topical administration.

A pharmaceutical composition can be administered to the eye via any suitable form or route including, for example, topical, oral, systemic, intravitreal, intracameral, subconjunctival, subtenon, retrobulbar, intraocular, posterior juxtascleral, periocular, subretinal, and suprachoroidal administration. The compositions can be administered by injecting the formulation in any part of the eye including anterior chamber, posterior chamber, vitreous chamber (intravitreal), retina proper, and/or subretinal space. The compositions can be delivered via a non-invasive method. Non-invasive modes of administering the formulation can include using a needleless injection device. Multiple administration routes can be employed for efficient delivery of the pharmaceutical compositions.

A pharmaceutical composition can be targeted to any suitable ocular cell including for example, endothelial cells such as vascular endothelial cells, cells of the retina such as retinal pigment epilthelium (RPE), corneal cells, fibroblasts, astrocytes, glial cells, pericytes, iris epithelial cells, cells of neural origin, ciliary epithelial cells, Muller cells, muscle cells surrounding and attached to the eye such as cells of the lateral rectus muscle, orbital fat cells, cells of the sclera and episclera, cells of the trabecular meshwork, and connective tissue cells.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

Pharmaceutical formulations for administration can include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, for example, sterile pyrogen-free water, before use.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compounds described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

The disclosed compositions can optionally comprise from about 0.001% to about 0.005% weight by volume pharmaceutically-acceptable preservatives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins1999), each of which is incorporated by reference in its entirety.

The disclosed methods include administration of a vector carrying a nucleic acid encoding a HPTPβ suppressor in combination with a pharmaceutically-acceptable carrier. The carrier can be selected to minimize any degradation of the active ingredient and to minimize any adverse side effects in the subject.

A vector described herein can be conveniently formulated into pharmaceutical compositions composed of one or more pharmaceutically-acceptable carriers. See e.g., Remington's Pharmaceutical Sciences, latest edition, by E. W. Martin Mack Pub. Co., Easton, Pa., incorporated by reference in its entirety, which discloses typical carriers and conventional methods of preparing pharmaceutical compositions. Such pharmaceutical can be carriers for administration of compositions to humans and non-humans, including solutions such as sterile water, saline, and buffered solutions at physiological pH. Pharmaceutical compositions can also include one or more additional active ingredients such as antimicrobial agents, anti-inflammatory agents, and anesthetics.

Non-limiting examples of pharmaceutically-acceptable carriers include saline, Ringer's solution, and dextrose solution. The pH of the solution can be from about 5 to about 8, and can be from about 7 to about 7.5. Further carriers include sustained release preparations such as semipermeable matrices of solid hydrophobic polymers containing the vector. The matrices can be in the form of shaped articles, for example, films, liposomes, microparticles, or microcapsules.

The disclosed methods relate to administering a nucleic acid encoding a HPTPβ suppressor as part of a pharmaceutical composition. Compositions suitable for topical administration can be used. In some embodiments, compositions of the invention can comprise a liquid comprising an active agent in solution, in suspension, or both. Liquid compositions can include gels. In one embodiment, the liquid composition is aqueous. Alternatively, the composition can take form of an ointment. In another embodiment, the composition is an in situ gellable aqueous composition. In iteration, the composition is an in situ gellable aqueous solution. Such a composition can comprise a gelling agent in a concentration effective to promote gelling upon contact with the eye or lacrimal fluid in the exterior of the eye. Aqueous compositions of the invention can have ophthalmically-compatible pH and osmolality. The composition can comprise an ophthalmic depot formulation comprising an active agent for subconjunctival administration. Microparticles comprising an active agent can be embedded in a biocompatible pharmaceutically-acceptable polymer or a lipid encapsulating agent. The depot formulations can be adapted to release all or substantially all the active material over an extended period of time. The polymer or lipid matrix, if present, can be adapted to degrade sufficiently to be transported from the site of administration after release of all or substantially all the active agent. The depot formulation can be a liquid formulation, comprising a pharmaceutical acceptable polymer and a dissolved or dispersed active agent. Upon injection, the polymer forms a depot at the injections site, for example, by gelifying or precipitating. The composition can comprise a solid article that can be inserted in a suitable location in the eye, such as between the eye and eyelid or in the conjuctival sac, where the article releases the active agent. Solid articles suitable for implantation in the eye in such fashion can comprise polymers and can be bioerodible or non-bioerodible.

Pharmaceutical formulations can include additional carriers, as well as thickeners, diluents, buffers, preservatives, and surface active agents in addition to the agents disclosed herein.

The pH of the disclosed composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

A vector of the disclosure can be incorporated into pharmaceutical compositions for administration to animal subjects, for example, humans. The vector or virions can be formulated in nontoxic, inert, pharmaceutically-acceptable aqueous carriers, for example, at a pH ranging from about 3 to about 8 or ranging from about 6 to 8. Such sterile compositions can comprise the vector containing the nucleic acid encoding the therapeutic molecule dissolved in an aqueous buffer having an acceptable pH upon reconstitution.

In some embodiments, the pharmaceutical composition provided herein comprise a therapeutically effective amount of a vector in admixture with a pharmaceutically-acceptable carrier and/or excipient, for example, saline, phosphate buffered saline, phosphate and amino acids, polymers, polyols, sugar, buffers, preservatives and other proteins. Illustrative agents include octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene, and glycol.

### Methods of Administration and Treatment Methods

Pharmaceutical compositions described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

### Amino Acids

Non-limiting examples of amino acids include hydrophilic amino acids, hydrophobic amino acids, charged amino acids, uncharged amino acids, acidic amino acids, basic amino acids, neutral amino acids, aromatic amino acids, aliphatic amino acids, natural amino acids, non-natural amino acids, synthetic amino acids, artificial amino acids, capped amino acids, genetically-encoded amino acids, non-genetically encoded amino acids, and amino acid analogues, homologues, and congeners. A non-natural amino0 acid can be, for example, an amino acid that is prepared chemically or expressed by tRNA synthetase technology. A non-limiting example of an achiral amino acid is glycine (G, Gly). Non-limiting examples of L-enantiomeric and D-enantiomeric amino acids are: alanine (A, Ala); arginine (R, Arg); asparagine (N, Asn); aspartic acid (D, Asp); cysteine (C, Cys); glutamic acid (E, Glu); glutamine (Q, Gln); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val). In some embodiments, conservative or non-conservative substitutions of amino acids are possible.

### Kits

The present disclosure further relates to kits containing the composition of the disclosure for use by medical or other trained personnel, as well as for use by trained subjects for delivery of the disclosed composition to a subject. A kit can comprise:
A) a composition comprising a vector comprising a nucleic acid encoding a HPTPβ suppressor; and
B) a carrier for delivering the composition to a subject.

The kits can be modified to fit the dosing regimen prescribed for the subject being treated. The following is a non-limiting example of a kit for use with a subject receiving a composition of the disclosure by an intraocular injection. This example provides a single injection of the composition once every 12 months.
A) an aqueous composition containing:
   a) an adeno-associated viral vector comprising a nucleic acid encoding a monoclonal antibody targeting HPTPβ extracellular domain; and
   b) a carrier system, comprising:
      i) a tonicity agent; and
      ii) water
      wherein the tonicity agent is present in an amount such that the such that the re-constituted formula comprises from about 0.5% to about 10% mass to volume of the tonicity agent; and
B) a component for delivering the aqueous composition.

The disclosed compositions can comprise, for example, from about 1.5% to about 90% mass by volume of a carrier system. The amount of carrier system present is based upon several different factors or choices made by the formulator, for example, the final concentration of the therapeutic agent and the amount of solubilizing agent.

Non-limiting examples of tonicity agents include dextrose, mannitol and glycerin. The formulator can utilize more than one tonicity agent when formulating the disclosed compositions. The tonicity agent can comprise from about 0.5% to about 5% weight by volume of the final composition.

The osmolarity of the disclosed compositions can be within any range chosen by the formulator, for example, from about 250 to about 350 mOsm/L, or from about 270 to about 310 mOsm/L.

The kit can further comprise a standard or control information so that a subject sample can be compared with the control to determine whether the test amount of recombinant virus is a therapeutic amount consistent with, for example, a reduction in angiogenesis. Optionally, the kit can further comprise devices for administration, such as a syringe, filter needle, extension tubing, cannula, and subretinal injector.

The composition of a kit can be administered to a subject. Non-limiting examples of routes of administration include intraocular, parenteral, and topical. Intraocular routes of administration can include, for example, intravitreal, intracameral, subconjunctival, subtenon, retrobulbar, intraocular, posterior juxtascleral, periocular, subretinal, and suprachoroidal. Delivery can be by, for example, syringe, needle, infusion pump, or injector. Syringes and injectors can be, for example, single-dose, multi-dose, fixed-dose, or variable-dose. Non-limiting examples of injectors include, pen injectors, auto-injectors, and electronic patch injector systems.

The kits can comprise suitable components for the administration of a composition of the invention to a subject. In some embodiments a composition of the invention is present in the kit as a unit dosage form. As such, the formulator can provide delivery devices having a higher concentration of compound and adjust the delivered volume to provide an amount of compound that is less than the amount in the entire solution. In another embodiment the kit comprises a delivery device that contains a sufficient amount of a composition to allow for administration of multiple doses from the delivery device.

A set of instructions can be included in any of the kits described herein. The instructions can relate to the dosing amount, timing of dosing, and reconstitution of the composition when the kit contains a dry composition, and methods of disposal of delivery vehicles and unused composition. The instructions can describe any therapy, compounds, excipients, or method of administration described herein.

### Methods

The invention provides compositions and methods for the treatment or prevention of diseases or conditions of the eye, for example, diabetic macular edema, age-related macular degeneration (wet form), choroidal neovascularization, diabetic retinopathy, ocular ischemia, uveitis, retinal vein occlusion (central or branch), ocular trauma, surgery induced edema, surgery induced neovascularization, cystoid macular edema, ocular ischemia, and uveitis. These diseases or conditions can be characterized by changes in the ocular vasculature, whether progressive or non-progressive, whether a result of an acute disease or condition, or a chronic disease or condition. These diseases can be characterized by an increased level of plasma Vascular Endothelial Growth Factor.

One embodiment of the present disclosure is a method of treating ocular neovascularization in a subject, the method comprising administering a pharmaceutically-effective amount of a nucleic acid encoding a HPTPβ suppressor. Another embodiment of the present disclosure is a method of treating ocular neovascularization in a subject, comprising administering an effective amount of a composition comprising a nucleic acid encoding a HPTPβ suppressor, and one or more pharmaceutically-acceptable excipient.

In some embodiments, the disclosed methods relate to the administration of a nucleic acid encoding fora HPTPβ suppressor, as well as compositions comprising a HPTPβ suppressor-encoding nucleic acid.

In some embodiments, the HPTPβ suppressor stabilizes the vasculature against leakage and neovascularization.

In one embodiment of the disclosed methods, a human subject with at least one visually impaired eye is treated with from about 10¹ to about 10¹⁸ vector genomes, for example, 10¹¹ vector genomes, of a recombinant vector comprising a nucleic acid encoding a HPTPβ suppressor via intraocular injection. The vector establishes a sustained production of HPTPβ suppressor inside host ocular cells. Improvement of clinical symptoms can be monitored, for example, indirect ophthalmoscopy, fundus photography, fluorescein angiopathy, electroretinography, external eye examination, slit lamp biomicroscopy, applanation tonometry, pachymetry, optical coherence tomography, or autorefaction. As described herein, the dosing can occur at any frequency determined by the administrator. Depending on the response, subsequent doses can be administered 12 to 18 months apart.

Diseases that are a direct or indirect result of diabetes include, inter alia, diabetic macular edema and diabetic retinopathy. The ocular vasculature of the diabetic becomes unstable over time leading to conditions such as non-proliferative retinopathy, macular edema, and proliferative retinopathy. As fluid leaks into the center of the macula, the part of the eye where sharp, straight-ahead vision occurs, the buildup of fluid and the associated protein begin to deposit on or under the macula. This deposit results in swelling that causes the subject's central vision gradually to become distorted. This condition is referred to as macular edema. Another condition that can occur is non-proliferative retinopathy in which vascular changes, such as microaneurysms, outside the macular region of the eye can be observed.

These conditions can be associated with diabetic proliferative retinopathy, which is characterized by increased neovascularization. These new blood vessels are fragile and are susceptible to bleeding. The result is scarring of the retina and occlusion or total blockage of the light pathway through the eye due to the over formation of new blood vessels. Subjects having diabetic macular edema often suffer from the non-proliferative stage of diabetic retinopathy; however, subjects often only begin to manifest macular edema at the onset of the proliferative stage.

Diabetic retinopathy is the most common cause of vision loss in working-aged Americans. Severe vision loss occurs due to tractional retinal detachments that complicate retinal neovascularization (NV), but the most common cause of moderate vision loss is diabetic macular edema (DME). Vascular endothelial growth factor (Vegf) is a hypoxia-regulated gene, and VEGF levels are increased in hypoxic or ischemic retina.

Angiopoietin-2 binds Tie2, but does not stimulate phosphorylation and therefore acts as an antagonist under most circumstances. In the eye, angiopoietin 2 is upregulated at sites of neovascularization and acts as a permissive factor for VEGF. Increased expression of VEGF in the retina does not stimulate sprouting of neovascularization from the superficial or intermediate capillary beds of the retina or the choriocapillaris, but does stimulate sprouting from the deep capillary bed where there is constitutive expression of angiopoietin 2. Co-expression of VEGF and angiopoietin 2 at the surface of the retina causes sprouting of neovascularization from the superficial retinal capillaries.

Regulation of Tie2 also occurs through HPTPβ. Mice deficient in VE-PTP (mouse orthologue of HPTPβ) die at E10 with severe defects in vascular remodeling and maturation of developing vasculature. RNAi-mediated silencing of HPTPβ in cultured human endothelial cells enhances Ang1-induced phosphorylation of Tie2 and survival-promoting activity, while hypoxia increases expression of HPTPβ and reduces Ang1-induced phosphorylation of Tie2.

Macular degeneration is a condition characterized by a gradual loss or impairment of eyesight due to cell and tissue degeneration of the yellow macular region in the center of the retina. Macular degeneration is often characterized as one of two types, non-exudative (dry form) or exudative (wet form). Although both types are bilateral and progressive, each type can reflect different pathological processes. The wet form of age-related macular degeneration (AMD) is the most common form of choroidal neovascularization and a leading cause of blindness in the elderly. AMD affects millions of Americans over the age of 60, and is the leading cause of new blindness among the elderly.

Currently-approved treatment for wet AMD involves repeat intraocular injections of anti-VEGF agents such as bevacizumab, ranibizumab, and aflibercept. These agents are rapidly cleared from the eye, therefore requiring repeat injections of relatively large amounts of the anti-VEGF agent at a frequency of about 4-8 weeks. Frequent intraocular injections and exposure of the eye to high concentrations of anti-VEGF agents carries a risk of adverse effects in the subject. The adverse effects can include infectious endophthalmitis, vitreous hemorrhage, retinal detachment, traumatic cataract, corneal abrasion, subconjunctival hemorrhage, and eyelid swelling. Moreover, in many subjects, the disease rapidly recurs if regular injections are interrupted.

The present disclosure provides a HPTPβ suppressor delivered by a suitable vector, for example, a recombinant viral system, to the retina of a subject for the treatment of neovascular retinal diseases.

Choroidal neovascular membrane (CNVM) is a problem that is related to a wide variety of retinal diseases, but is most commonly linked to age-related macular degeneration. With CNVM, abnormal blood vessels stemming from the choroid (the blood vessel-rich tissue layer just beneath the retina) grow up through the retinal layers. These new vessels are very fragile and break easily, causing blood and fluid to pool within the layers of the retina.

Diabetes (diabetes mellitus) is a metabolic disease caused by the inability of the pancreas to produce insulin or to use the insulin that is produced. The most common types of diabetes are type 1 diabetes (often referred to as Juvenile Onset Diabetes Mellitus) and type 2 diabetes (often referred to as Adult Onset Diabetes Mellitus). Type 1 diabetes results from the body's failure to produce insulin due to loss of insulin producing cells, and presently requires the person to inject insulin. Type 2 diabetes generally results from insulin resistance, a condition in which cells fail to use insulin properly.

Diabetes can be correlated to a large number of other conditions, including conditions or diseases of the eye including diabetic retinopathy (DR) and diabetic macular edema (DME) which are leading causes of vision loss and blindness in most developed countries. The increasing number of individuals with diabetes worldwide suggests that DR and DME continues to be major contributors to vision loss and associated functional impairment for years to come.

Diabetic retinopathy is a complication of diabetes that results from damage to the blood vessels of the light-sensitive tissue at the back of the eye (retina). At first, diabetic retinopathy can cause no symptoms or only mild vision problems. Eventually diabetic retinopathy can result in blindness. Diabetic retinopathy can develop in anyone who has type 1 diabetes or type 2 diabetes.

At the earliest stage of non-proliferative retinopathy, microaneurysms occur in the retina's tiny blood vessels. As the disease progresses, more of these blood vessels become damaged or blocked and these areas of the retina send signals into the regional tissue to grow new blood vessels for nourishment. This stage is called proliferative retinopathy. The new blood vessels grow along the retina and along the surface of the clear, vitreous gel that fills the inside of the eye. The vessels have thin, fragile walls and without timely treatment, the new blood vessels can leak blood, for example, whole blood or some constituents thereof, and can result in severe vision loss and even blindness. Also, fluid can leak into the center of the macula, the part of the eye where sharp, straight-ahead vision occurs. The fluid and the associated protein begin to deposit on or under the macula swell the subject's central vision becomes distorted. This condition is called macular edema and can occur at any stage of diabetic retinopathy, but is more likely to occur as the disease progresses. About half of the people with proliferative retinopathy also have macular edema.

Uveitis is a condition in which the uvea becomes inflamed. The eye is hollow on the inside with three different layers of tissue surrounding a central cavity. The outermost is the sclera (white coat of the eye) and the innermost is the retina. The middle layer between the sclera and the retina is called the uvea. The uvea contains many of the blood vessels that nourish the eye. Complications of uveitis include glaucoma, cataracts or new blood vessel formation (neovascularization).

Ocular trauma is any sort of physical or chemical injury to the eye. Ocular trauma can affect anyone and major symptoms include redness or pain in the affected eye. Neither symptom can occur if tiny projectiles are the cause of the trauma.

Surgery-induced edema is the development of swelling in the eye tissues following surgery on the retina or other part of the eye. Cystoid macular edema (CME) is an example of this phenomenon. CME can occur not only in people who have had cataract surgery, but also those with diabetes, retinitis pigmentosa, AMD, or conditions that cause chronic inflammation in the eye. The major symptoms of CME are blurred or decreased central vision.

Ocular ischemic syndrome (OIS) encompasses the signs and symptoms that result from chronic vascular insufficiency. The condition is caused by ocular hypoperfusion due to occlusion or stenosis of the common or internal carotid arteries. OIS generally affects subjects that are between the ages of 50-80 and can have systemic diseases such as hypertension or diabetes. The major symptoms of OIS are orbital pain, vision loss, changes of the visual field, asymmetric cataract, and sluggish reaction to light, among a variety of other symptoms.

Retinal vein occlusion (RVO) is the most common retinal vascular disease after diabetic retinopathy. Depending on the area of retinal venous drainage effectively occluded, the condition is broadly classified as central retinal vein occlusion (CRVO), hemispheric retinal vein occlusion (HRVO), or branch retinal vein occlusion (BRVO). Presentation of RVO is with variable painless visual loss with any combination of fundal findings consisting of retinal vascular tortuosity, retinal hemorrhages (blot and flame shaped), cotton wool spots, optic disc swelling and macular edema. In a CRVO, retinal hemorrhages can be found in all four quadrants of the fundus, while these are restricted to either the superior or inferior fundal hemisphere in a HRVO. In a BRVO, hemorrhages are largely localized to the area drained by the occluded branch retinal vein. Vision loss occurs secondary to macular edema or ischemia.

Angiogenesis, the process of creating new blood vessels from pre-existing vessels, is essential to a wide range of physiological and pathological events including embryological development, menstruation, wound healing, and tumor growth. Most, if not all, tumors require angiogenesis to grow and proliferate. VEGF is a major factor in angiogenesis and can increase vessel permeability and capillary number.

Compositions of the disclosure act to stabilize ocular vasculature and, in some embodiments, an agent of the disclosure can counteract the stimulation caused by VEGF and other inflammatory agents that can be present in the diseased retina. In some embodiments, administration of a nucleic acid encoding a HPTPβ suppressor to a subject can be used to maintain the level of disease reversal after administration of anti-VEGF drugs to the subject have been withdrawn.

Recombinant viruses can be produced by any suitable methods. For example, recombinant viruses can be generated through transfection of insect cells via recombinant baculovirus. In some embodiments, recombinant baculovirus can be generated as an intermediate, whereby the baculovirus can contain sequences necessary for the generation of other viruses such as AAV or rAAV2 viruses. In some embodiments, one or more baculoviruses can be used in the generation of recombinant viruses used for the composition and methods of treatment of this disclosure. In some embodiments, insect cells such as Sf9, High-Five or Sf21 cell lines can be used. Cell lines can be generated using transient methods, i.e. infection with transgenes not stably integrated. Cell lines can be generated through the generation of stable cell lines i.e. infection with transgenes stably integrated into the host cell genome. Pharmaceutical compositions provided herein can be manufactured using human embryonic kidney 293 (HEK293) cells, suspension-adapted HEK293 cells, baculovirus expression system (BVES) in insect cells, herpes-helper virus, producer-clone methods, or Ad-AAV.

Any suitable method can be used in the biochemical purification of recombinant viruses for use in a pharmaceutical composition as described herein. Recombinant viruses can be harvested directly from cells, or from the culture media surrounding host cells. Virus can be purified using various biochemical methods, such as gel filtration, filtration, chromatography, affinity purification, gradient ultracentrifugation, or size exclusion methods. Recombinant virus can be tested for content, for example, identity, purity, or potency, for example, activity, using any suitable methods, before formulation into a pharmaceutical composition. Methods can include immunoassays, ELISA, SDS-PAGE, western blot, Northern blot, Southern blot or PCR, and HUVEC assays.

### Intraocular Delivery

Disclosed herein are methods for intraocular delivery of compositions of the invention to a subject having a disease or condition as disclosed herein. The delivery method can include an invasive method for direct delivery of the composition to ocular cells. In one embodiment, a liquid pharmaceutical composition comprising the vector is delivered via a subretinal injection. In another embodiment, a liquid pharmaceutical composition comprising the vector is delivered via an intravitreal injection. In some embodiments, the composition is delivered via multiple administration routes, for example, subretinal and/or intravitreous, to increase efficiency of the vector delivery. In some embodiments, the subretinal and/or intravitreal injection is preceded by a vitrectomy.

The intraocular injection can be performed over any interval of time to optimize efficiency of delivery and/or to minimize or avoid damage to surrounding tissue. The interval of time for the intraocular injection can be from, for example, about 1 minute to about 60 minutes, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes.

The intraocular injection can be performed at any rate. The rate of intraocular injection can be from, for example, about 1 μL/min to about 200 μL/min, about 1 μL/min to about 10 μL/min, about 10 μL/min to about 20 μL/min, about 20 μL/min to about 30 μL/min, about 30 μL/min to about 40 μL/min, about 40 μL/min to about 50 μL/min, about 50 μL/min to about 60 μL/min, about 60 μL/min to about 70 μL/min, about 70 μL/min to about 80 μL/min, about 80 μL/min to about 90 μL/min, about 90 μL/min to about 100 μL/min, about 100 μL/min to about 110 μL/min, about 110 μL/min to about 120 μL/min, about 120 μL/min to about 130 μL/min, about 130 μL/min to about 140 μL/min, about 140 μL/min to about 150 μL/min, about 150 μL/min to about 160 μL/min, about 160 μL/min to about 170 μL/min, about 170 μL/min to about 180 μL/min, about 180 μL/min to about 190 μL/min, or about 190 μL/min to about 200 μL/min.

### Treatment of Subjects

In some embodiments, a single administration of the composition of the disclosure in a subject having a disease or condition as disclosed herein results in sustained intraocular expression of a HPTPβ suppressor at a level sufficient for long-term suppression of ocular neovascularization.

For example, the level of HPTPβ suppressor produced in a host ocular cell can be at least 100 pg/mL, at least 200 pg/mL, at least 300 pg/mL, at least 400 pg/mL, at least 500 pg/mL, at least 600 pg/mL, at least 00 pg/mL, at least 800 pg/mL, at least 900 pg/mL, at least 1000 pg/mL, at least 2000 pg/mL, at least 3000 pg/mL, at least 4000 pg/mL, at least 5000 pg/mL, at least 6000 pg/mL, at least 7000 pg/mL, at least 8000 pg/mL, at least 9000 pg/mL or at least 10,000 pg/mL. The level of HPTPβ suppressor produced in host ocular cell can be at most 100 pg/mL, at most 200 pg/mL, at most 300 pg/mL, at most 400 pg/mL, at most 500 pg/mL, at most 600 pg/mL, at most 700 pg/mL, at most 800 pg/mL, at most 900 pg/mL, at most 1000 pg/mL, at most 2000 pg/mL, at most 3000 pg/mL, at most 4000 pg/mL, at most 5000 pg/mL, at most 6000 pg/mL, at most 7000 pg/mL, at most 8000 pg/mL, at most 9000 pg/mL or at most 10,000 pg/mL.

Protein levels can be measured at least about 0.1, at least about 0.2, at least about 0.3, at least about 0.4, at least about 0.5, at least about 0.6, at least about 0.7, at least about 0.8, at least about 0.9, at least about 1, at least about 2, at least about 3, at least about 4, at least about 5, at least about 6, at least about 7, at least about 14, at least about 21, at least about 30, at least about 50, at least about 75, at least about 100, at least about 125, at least about 150, at least about 175, at least about 200, at least about 225, at least about 250, at least about 275, at least about 300, at least about 325, at least about 350, or at least about 365 days after administering a pharmaceutical composition of the disclosure. Protein levels can be measured at most about 0.1, at most about 0.2, at most about 0.3, at most about 0.4, at most about 0.5, at most about 0.6, at most about 0.7, at most about 0.8, at most about 0.9, at most about 1, at most about 2, at most about 3, at most about 4, at most about 5, at most about 6, at most about 7, at most about 14, at most about 21, at most about 30, at most about 50, at most about 75, at most about 100, at most about 125, at most about 150, at most about 175, at most about 200, at most about 225, at most about 250, at most about 275, at most about 300, at most about 325, at most about 350, or at most about 365 days after administering a pharmaceutical composition of the disclosure.

### Central Foveal Thickness

Also disclosed herein are methods for decreasing the Central Foveal Thickness (CFT) in a subject having a disease or condition as disclosed herein. The method comprises administering to an eye a nucleic acid encoding a HPTPβ suppressor, wherein the administration of the nucleic acid can be conducted in any manner desired by the administrator, for example, as further described herein.

The level of decrease in Central Foveal Thickness can be for example, from about 50 μm to about 1000 μm. The level of decrease in Central Foveal Thickness can be for example, from about 50 μm to about 500 μm, from about 50 μm to about 750 μm, from about 150 μm to about 500 μm, from about 200 μm to about 500 μm, from about 200 μm to about 1000 μm, from about 250 μm to about 650 μm, or from about 400 μm to about 700 μm.

### Visual Acuity

Further disclosed herein are methods for increasing the visual acuity of a subject having a disease or condition as disclosed herein.

Visual acuity (VA) is acuteness or clearness of vision, which is dependent on the sharpness of the retinal focus within the eye and the sensitivity of the interpretative faculty of the brain. Visual acuity is a measure of the spatial resolution of the visual processing system. VA is tested by requiring the person whose vision is being tested to identify characters typically numbers or letters on a chart from a set distance. Chart characters are represented as black symbols against a white background. The distance between the person's eyes and the testing chart is set at a sufficient distance to approximate infinity in the way the lens attempts to focus. Twenty feet, or six meters, is essentially infinity from an optical perspective. In the present disclosure, an improvement in visual acuity was assessed by an increase in the number of letters read from the chart.

One non-limiting test for measuring Visual Acuity is the use of the ESV-3000 ETDRS testing device and self-calibrated test lighting. The ESV-3000 device incorporates LED light source technology. The auto-calibration circuitry constantly monitors the LED light source and calibrates the test luminance to 85 cd/m² or 3 cd/m².

Although designed for clinical trials where large-format ETDRS testing (up to 20/200) is performed at 4 meters, the device can be used in a non-research setting, i.e., hospital or clinic where ocular disease monitoring is conducted. To evaluate ETDRS properly, the test should be conducted under standardized lighting conditions, for, example, photopic test level of 85 cd/m². Scoring of visual acuity can be accomplished in any manner chosen by the monitor. After providing a baseline evaluation, the increase or decrease in the number of letters that can be identified by the test subject provides a measure of sight increase or decrease during treatment.

Disclosed herein is a method for increasing visual acuity in a subject having a disease or condition of the eye as disclosed herein. This method comprises administering to a subject having the disease or condition of the eye, a nucleic acid encoding a HPTPβ suppressor, wherein the administration of the nucleic acid can be conducted in any manner desired by the administrator, for example, as further described herein.

In one embodiment, the disclosure provides a method for increasing the number of letters recognizable by a treated eye. The increase in the number of letters recognized by a treated eye can be, for example, from about 1 to about 30 letters, from about 5 to about 25 letters, from about 5 to about 20 letters, from about 5 to about 15 letters, from about 5 to about 10 letters, from about 10 to about 25 letters, from about 15 to about 25 letters, or from about 20 to about 25 letters. The increase in visual acuity can be about 1 letter, about 5 letters, about 10 letters, about 15 letters, about 20 letters, or about 25 letters.

### EXAMPLES

### Example 1

### Identification and Characterization of a VE-PTP Extracellular Domain Binding Agent

VE-PTP (SEQ ID NO. 15) is the mouse orthologue of HPTPβ. Antibodies to the VE-PTP extracellular domain were identified and characterized as summarized below.

### A. Generation of Antibodies to VE-PTP Extracellular Domain Protein (VE-PTP-ECD)

VE-PTP-Fc fusion protein was constructed such that the first 8 fibronectin type III-like repeats ending with the amino acid proline at position 732 of VE-PTP (SEQ ID NO. 16) were fused in frame with the Fc portion of human IgG1, starting with amino acid proline at position 239. This construct cloned into pcDNA3 was stably transfected into CHO cells, and the fusion protein was purified by protein A Sepharose™ affinity purification.

The antibody was generated by immunizing rats with the VE-PTP-Fc fusion protein. Immunization, hybridoma-fusion, and screening were conducted using standard methods.

### B. Anti-VE-PTP-ECD Activity Studies in Mice Eyes

### Laser-Induced Choroidal Neovascularization Model

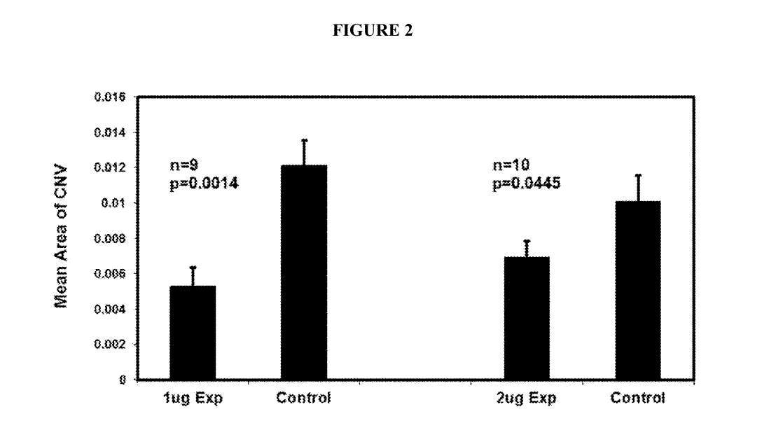

Laser-induced choroidal neovascularization model is considered to represent a model of neovascular age-related macular degeneration. Adult C57BL/6 mice had laser-induced rupture of Bruch's membrane in three locations in each eye and were then given intravitreal injections of 1 or 2 μg of an anti-VE-PTP-ECD antibody (IgG2a) in one eye and vehicle (5% dextrose) in the fellow eye. These treatments were repeated on day 7. Fourteen days after laser, the mice were perfused with fluorescein-labeled dextran (2×10⁶ average MW) and the extent of neovascularization was assessed in choroidal flat mounts by fluorescence microscopy. The area of CNV at each Bruch's membrane rupture site was measured by image analysis by an observer masked with respect to treatment group. The area of CNV is the average of the three rupture sites in one eye. As shown in FIG. 2, treatment with the anti-VE-PTP-ECD antibody significantly reduced choroidal neovascularization at both 1 and 2 μg doses versus treatment with vehicle control.

### Ischemic Retinopathy Model

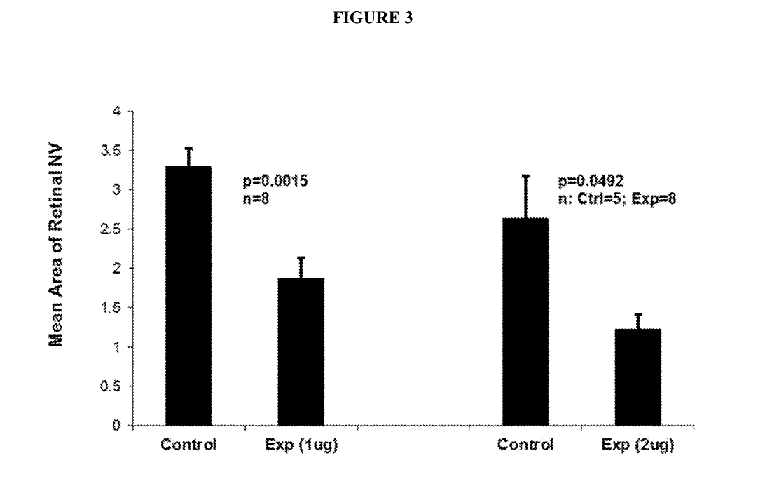

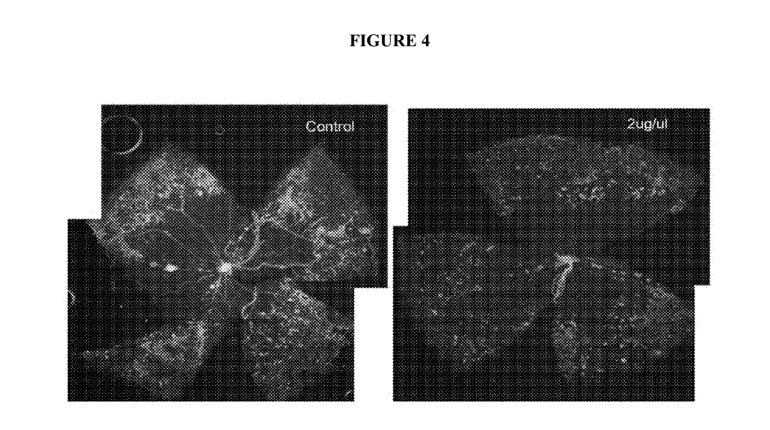

The oxygen-induced ischemic retinopathy model represents a model of proliferative diabetic retinopathy. C57BL/6 mice at postnatal day 7 (P7) and their mothers were placed in an airtight chamber and exposed to hyperoxia (75±3% oxygen) for five days. Oxygen was continuously monitored with a PROOX model 110 oxygen controller. On P12, mice were returned to ambient air. Under a dissecting microscope, a Harvard Pump Microinjection System and pulled glass pipettes were used to deliver an intravitreal injection of 1 or 2 μtg of an anti-VE-PTP-ECD antibody in one eye and vehicle in the fellow eye. At P17, the area of NV on the surface of the retina was measured at P17. Briefly, mice were given an intraocular injection of 0.5 μg rat anti-mouse PECAM antibody. Twelve hours later, the mice were euthanized and the eyes were fixed in 10% formalin. The retinas were dissected, incubated for 40 minutes in 1:500 goat anti-rat IgG conjugated with Alexa Fluor® 488 (Invitrogen™, Carlsbad, Calif.), washed, and whole mounted. An observer masked with respect to treatment group examined the slides with a fluorescence microscope and measured the area of NV per retina by computerized image analysis using Image-Pro Plus software. FIG. 3 shows that treatment with the anti-VE-PTP-ECD antibody significantly reduced retinal neovascularization at both 1 and 2 μg doses versus treatment with vehicle control. FIG. 4 shows representative retinal whole mounts from a mouse treated with vehicle versus a mouse treated with 2 μg of the anti-VE-PTP-ECD antibody.

### Example 2

### Identification and Characterization of HPTPβ Extracellular Domain Binding Agents

Antibodies to the HPTPβ extracellular domain (SEQ ID NO. 17) are identified and characterized as summarized below.

### Generation of Antibodies to the HPTPβ Extracellular Domain

An HPTPβ fusion protein is constructed such that the extracellular domain (SEQ ID NO. 17) is fused in frame with the Fc portion of human IgG1, starting with amino acid proline at position 239 (herein referred to as HPTPβ-ECD-Fc). This construct is cloned into pcDNA3 (Invitrogen™, Carlsbad, Calif.) and stably transfected into CHO cells, and the fusion protein is purified by protein A Sepharose™ affinity purification.

The antibody is generated by immunizing mice with the HPTPβ-ECD-Fc fusion protein. Immunization, hybridoma-fusion, and screening are conducted using standard methods.

Generation of Antibodies to the HPTPβ first FN3 Repeat

An HPTPβ fusion protein is constructed such that the first FN3 repeat (SEQ ID NO. 18) is fused in frame with the Fc portion of human IgG1, starting with amino acid proline at position 239 (herein referred to as HPTPβ-FN3.1-Fc). This construct is cloned into pcDNA3 and stably transfected into CHO cells, and the fusion protein is purified by protein A Sepharose™ affinity purification.

The antibody is generated by immunizing mice with the HPTPβ-FN3.1-Fc fusion protein. Immunization, hybridoma-fusion, and screening are conducted using standard methods.

### Example 3

### Sequence Analysis of a HPTPβ Extracellular Domain Binding Agent

The sequence encoding antibody R15E6 was determined; the procedure and results are summarized below.

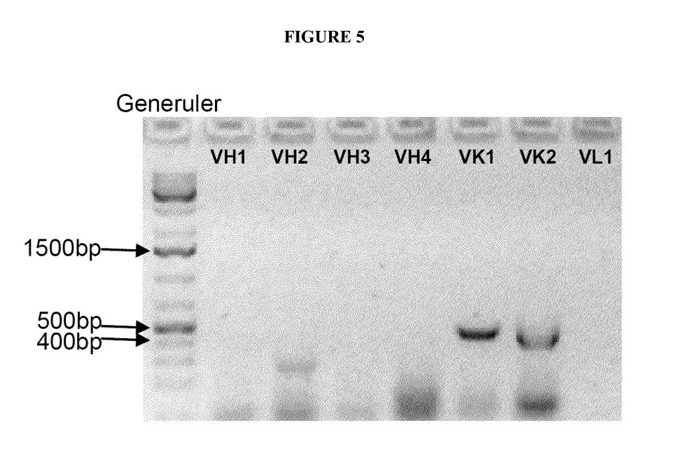

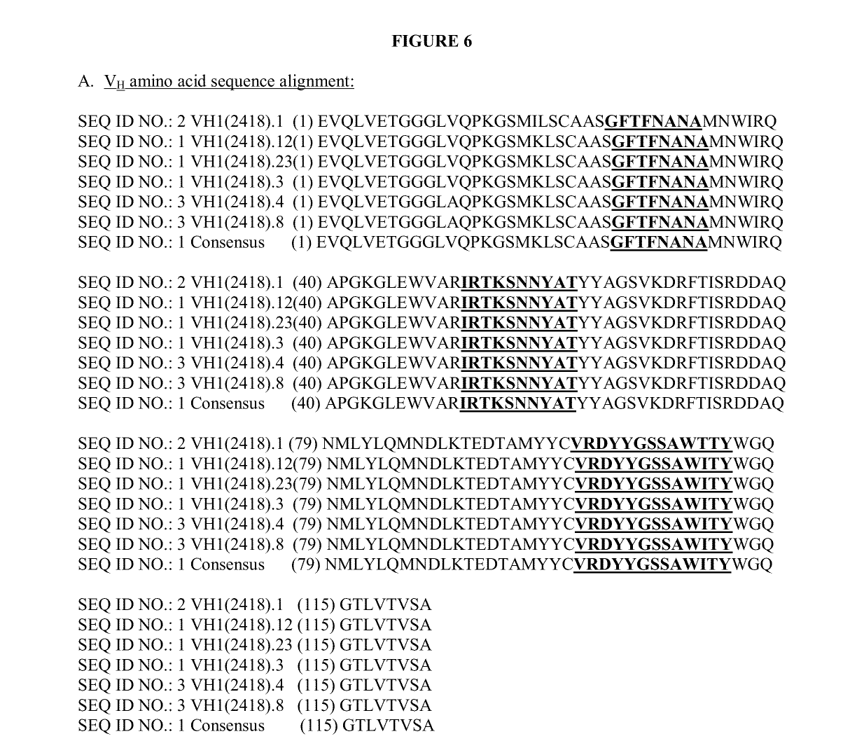

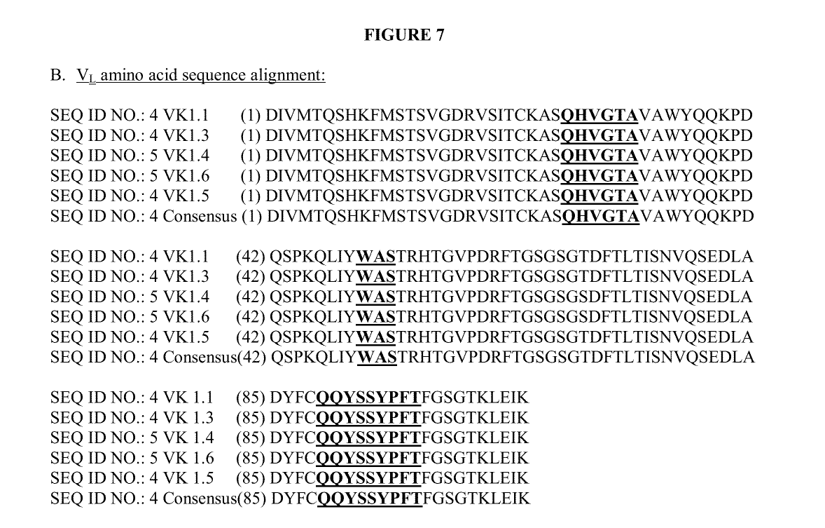

Total RNA was extracted from hybridoma cell pellets. Reverse transcription with an oligo(dT) primer was performed to create cDNA from the RNA. The V_{H} and V_{L} regions of R15E6 were amplified from the cDNA using variable domain primers to generate the bands in FIG. 5. The V_{H} and V_{L} products were cloned into the plasmid pCR2.1, transformed into E. coli, and screened by PCR for positive transformants. Positive transformants were analyzed by DNA sequencing, Resulting DNA sequences were compared to determine individual and consensus amino acid sequences of the V_{H} and V_{L} regions. FIGS. 6 and 7 show the individual and consensus amino acid sequence results for the V_{H} and V_{L} regions, respectively.

From the sequence analysis, consensus amino acid and DNA sequences were determined for the V_{H} (SEQ ID NO.: 1 and SEQ ID NO.: 11, respectively) and V_{L} (SEQ ID NO.: 4 and SEQ NO.: 12, respectively) regions. Variant sequences were also determined for the V_{H} (SEQ ID NO.: 2-3) and V_{L} (SEQ ID NO.: 5) regions. From these regions CDRs were determined for the V_{H} (SEQ ID NO.: 3-5) and V_{L}, (SEQ ID NO.: 6, WAS, and SEQ ID NO.: 7) regions. FIGS. 6 and 7 show the V_{H} and V_{L} consensus amino acid sequences, including the CDRs.

### Example 4

### Generation of a Humanized HPTPβ Extracellular Domain Binding Agent

From the sequence analysis in Example 3, a humanized antibody that binds and suppresses HPTPβ is generated. In one example, the CDR-grafting approach is used to generate the humanized antibody. The R15E6 consensus CDR sequences from FIGS. 6 and 7 are inserted into human immunoglobulin sequence templates to generate modified human antibody sequences that contain the CDRs for binding HPTPβ.

Upon generation of the recombinant sequence for the human antibody that binds HPTPβ, the recombinant sequences are cloned from the original vectors into other vectors for antibody production or gene delivery.

### Example 5

### Construction of a Recombinant Adeno-Associated Viral (rAAV) Vector Comprising a Nucleic Acid Sequence Encoding a Humanized Anti-HPTPβ Antibody

A recombinant adeno-associated viral (rAAV) vector of serotype 2 is used for cloning the humanized anti-HPTPβ antibody (discussed in Example 4). The rAAV vector comprises an expression cassette with a multiple cloning site, a cytomegalovirus (CMV) promoter, an internal ribosome entry site (IRES), and a simian virus (SV)40 polyadenylation site. The entire cassette is flanked by inverted terminal repeat sequences from AAV serotype 2. cDNA for anti-HPTPβ monoclonal antibody heavy and light chains is cloned into the multiple cloning site of rAAV to generate the recombinant adeno-associated viral vector, rAAV.HPTPβmab, which encodes for anti-HPTPβ monoclonal antibody when expressed.

rAAV.HPTPβmab vector is produced in Human Embryonic Kidney (HEK) 293 cells, which are maintained in Dulbecco's modified Eagles medium (DMEM), supplemented with 5% fetal bovine serum (FBS), 100 units/mL penicillin, 100 μg/mL streptomycin in 37° C. incubator with 5% CO₂. The cells are plated at 30-40% confluence in CellSTACK® (Corning®) 24 hours before transfection (70-80% confluence when transfected). The cells are co-transfected with 0.6 mg of the rAAV.HPTPβmab expression cassette plasmid comprising a cDNA encoding the anti-HPTPβ antibody, 0.6 mg packaging plasmid comprising a nucleic acid sequence encoding the AAV2 rep protein, and 1.8 mg adenovirus helper plasmid. After incubation at 37° C. for 72 hours, cells are harvested and lysed by multiple (at least three) freeze/thaw cycles. The cell lysate is treated with 50 U/mL of Benzonase® followed by iodixanol gradient centrifugation and QHP anion-exchange chromatography to purify the rAAV.HPTPβmab vector. The purified eluate is concentrated with a Centricon® Plus-20 100K concentrator. Vector genome titer is determined by quantitative TaqMan® real-time PCR analysis using a CMV promoter-specific primer-probe set. rAAV.HPTPβmab vector genome titers can range from 1.0×10¹-1×10¹⁸ vector genomes/mL.

### Example 6

### Treatment of Ocular Diseases in a Human Subject with a Recombinant Adeno-Associated Viral Vector Encoding a Hptpβ Suppressor

A dose of 100 μL buffer containing 10¹¹ vector genomes of the adeno-associated viral vector rAAV.HPTPβmab (described in Example 5) is administered via intraocular injection to one or both eyes of a human subject with visual acuity loss due to diabetic macular edema. The vector is injected at a rate of 100 μL/min over a period of 5 minutes. The injection is carried out using a cannula with a bore size of about 27-45 gauge, for example, using a 32-gauge needle. The injection delivers the vector directly in the subretinal space within the central retina of the subject.

Optical Coherence Tomography (OCT) is performed to monitor center point retinal thickness and fluid leakage in the retina of subjects. Multiple (at least 10) radial scans through the macula, each approximately 6 mm in length, are taken and OCT images/scans are collected at each specified visit post-treatment, for example, on Day 0 [baseline], Day 15, Day 30, Day 60, Day 180, and Day 365. The OCT images are evaluated for the presence of intraretinal fluid by a masked reader and the central retinal thickness is measured using Heidelberg Heyex SD-OCT software. The mean change in central retinal thickness using baseline at Day 0 is calculated.

Best corrected visual acuity is measured by a standard vision test at regular intervals post-treatment for example, on Day 0 [baseline], Day 15, Day 30, Day 60, Day 180, and Day 365. The mean change in visual acuity using baseline at Day 0 is calculated.

A mean change in visual acuity and central retinal thickness over time following treatment are used to assess the efficacy of the compositions and methods of the disclosure in treating diabetic macular edema.

### Safety Studies

Opthalmic examinations are conducted over a period of three months post-intraocular injection to assess retinal toxicity and inflammation.

Levels of anti-HPTPβ monoclonal antibody are measured in the subject's tears, blood, saliva and urine samples at regular intervals post-injection, for example, on Day 0 [baseline], Day 15, Day 30, Day 60, Day 180, and Day 365, using a HPTPβ specific enzyme-linked immunosorbent assay (ELISA).

The presence of the recombinant vector in the subject's tears, blood, saliva and urine samples is measured at regular intervals post-injection, for example, on Day 0 [baseline], Day 15, Day 30, Day 60, Day 180, and Day 365, using AAV2 capsid protein quantitation by ELISA.

Peripheral blood lymphocytes are isolated from the subject's blood sample for flow cytometry to assess immune cell subset response post-injection. Blood biochemistry, complete blood count, and T-cell response are measured.

### Example 7

### Baseline Safety and Efficacy Study for Determining the Effectiveness of the Disclosed Methods for Treating Ocular Diseases

The following experiment is conducted to evaluate the outcome of a composition of the disclosure in treating human subjects with ocular diseases.

Purpose: to evaluate the outcome of treating human subjects with visual acuity loss due to diabetic macular edema (central retinal thickness (CRT) of more than 325 microns and best corrected visual acuity less than 70 letters) with the recombinant adeno-associated viral vector rAAV.HPTPβmAb (described in Example 5).

Rationale: administration of a composition of the disclosure can establish production of a therapeutically effective amount of a HPTPβ suppressor in resident ocular cells.

Methods: a study is designed with some, or all, of the following experimental arms.
1) experimental arm 1: a dose of rAAV.HPTPβmAb, from about 10⁹ vector genomes to about 10¹³ vector genomes in 200 μL buffer, for example, 200 μL buffer containing 10¹¹ vector genomes, is administered via intraocular injection at 365 day intervals for 60 months to a first group of experimental subjects.
2) control arm 2: a first control composition, for example, empty rAAV vector, is administered via intraocular injection at 365 day intervals for 60 months to a second group of control subjects.
3) control arm 3: a second control composition, for example, PBS or an alternative buffer is administered via intraocular injection at 365 day intervals for 60 months to a third group of control subjects.

Retinal thickness and best corrected visual acuity are assessed at regular intervals post treatment, for example, on Day 0 [baseline], Day 15, Day 30, Day 60, Day 180, and Day 365. Student's t test is used to assess the significance of the effects of experimental and control arms in treating diabetic macular edema. The main efficacy outcome for the study is treatment of diabetic macular edema as measured by evaluating a change in visual acuity and central retinal thickness over a long-term period, for example, a time frame of 1 year, following administration of the compositions and methods of the disclosure.

### EMBODIMENTS

Embodiment 1. A pharmaceutical composition comprising a nucleic acid, wherein the nucleic acid is carried by a vector, wherein the nucleic acid encodes a tyrosine phosphatase suppressor.

Embodiment 2. The pharmaceutical composition of embodiment 1, wherein the tyrosine phosphatase is HPTPβ.

Embodiment 3. The pharmaceutical composition of any one of embodiments 1-2, wherein the tyrosine phosphatase suppressor is a monoclonal antibody or an antigen-binding fragment thereof.

Embodiment 4. The pharmaceutical composition of any one of embodiments 1-2, wherein the vector is a viral vector.

Embodiment 5. The pharmaceutical composition of embodiment 4, wherein the viral vector is an adenovirus-associated viral vector.

Embodiment 6. The pharmaceutical composition of any one of embodiments 1-5, wherein the tyrosine phosphatase suppressor binds an extracellular domain of HPTPβ.

Embodiment 7. The pharmaceutical composition of any one of embodiments 1-6, wherein the tyrosine phosphatase suppressor binds the first FN3 repeat of an extracellular domain of HPTPβ.

Embodiment 8. The pharmaceutical composition of any one of embodiments 1-7, wherein the tyrosine phosphatase suppressor binds a sequence with at least 90% homology to SEQ ID NO.: 17.

Embodiment 9. The pharmaceutical composition of any one of embodiments 3-8, wherein the monoclonal antibody or the antigen-binding fragment thereof comprises a heavy chain variable region having at least 90% homology to SEQ ID NO.: 1.

Embodiment 10. The pharmaceutical composition of any one of embodiments 3-9, wherein the monoclonal antibody or the antigen-binding fragment thereof comprises a light chain variable region having at least 90% homology to SEQ ID NO.: 4.

Embodiment 11. The pharmaceutical composition of any one of embodiments 1-10, comprising from about 1 ng to about 1 mg of the vector.

Embodiment 12. The pharmaceutical composition of any one of embodiments 1-11, the pharmaceutical composition further comprising a pharmaceutically-acceptable excipient, wherein the pharmaceutical composition is in a unit dosage form.

Embodiment 13. A pharmaceutical composition comprising a nucleic acid, wherein the nucleic acid is carried by a vector, wherein the nucleic acid encodes a Tie2 activator.

Embodiment 14. The pharmaceutical composition of embodiment 13, wherein the vector is a viral vector.

Embodiment 15. The pharmaceutical composition of embodiment 14, wherein the viral vector is an adenovirus-associated viral vector.

Embodiment 16. The pharmaceutical composition of any one of embodiments 13-15, wherein the Tie2 activator is a monoclonal antibody or an antigen-binding fragment thereof.

Embodiment 17. The pharmaceutical composition of embodiment 16, wherein the monoclonal antibody or antigen-binding fragment thereof binds to a tyrosine phosphatase.

Embodiment 18. The pharmaceutical composition of embodiment 17, wherein the tyrosine phosphatase is HPTPβ.

Embodiment 19. The pharmaceutical composition of any one of embodiments 13-18, wherein the Tie2 activator binds an extracellular domain of HPTPβ3.

Embodiment 20. The pharmaceutical composition of any one of embodiments 13-19, wherein the Tie2 activator binds the first FN3 repeat of an extracellular domain of HPTPβ.

Embodiment 21. The pharmaceutical composition of any one of embodiments 13-20, wherein the Tie-2 activator binds a sequence with at least 90% homology to SEQ ID NO.: 17.

Embodiment 22. The pharmaceutical composition of any one of embodiments 16-21, wherein the monoclonal antibody or the antigen-binding fragment thereof comprises a heavy chain variable region having at least 90% homology to SEQ ID NO.: 1.

Embodiment 23. The pharmaceutical composition of any one of embodiments 16-22, wherein the monoclonal antibody or the antigen-binding fragment thereof comprises a light chain variable region having at least 90% homology to SEQ ID NO.: 4.

Embodiment 24. The pharmaceutical composition of any one of embodiments 13-23, comprising from about 1 ng to about 1 mg of the vector.

Embodiment 25. The pharmaceutical composition of one of embodiments 13-24, the pharmaceutical composition further comprising a pharmaceutically-acceptable excipient, wherein the pharmaceutical composition is in a unit dosage form.

Embodiment 26. A method for treating a condition in a human in need thereof, the method comprising administering to the human a therapeutically-effective amount of a pharmaceutical composition comprising any one of embodiments 1-25.

Embodiment 27. The method of embodiment 26, wherein the condition is an ocular condition.

Embodiment 28. The method of any one of embodiments 26-27, wherein the composition is administered by intraocular injection.

Embodiment 29. The method of any one of embodiments 26-28, wherein treating the condition comprises reducing neovascularization in an eye.

Embodiment 30. The method of any one of embodiments 26-29, wherein treating the condition comprises reducing vascular leak in an eye.

Embodiment 31. The method of any one of embodiments 26-30, wherein treating the condition comprises increasing vascular stability in an eye.

Embodiment 32. The method of any one of embodiments 26-31, wherein the condition is a wet age-related macular degeneration.

Embodiment 33. The method of any one of embodiments 26-31, wherein the condition is retinal vein occlusion.

Embodiment 34. The method of any one of embodiments 26-31, wherein the condition is diabetic macular edema.

Embodiment 35. A method of administering a Tie2 activator to a cell, the method comprising contacting a cell with a nucleic acid, wherein the nucleic acid is carried by a vector, wherein the nucleic acid encodes a Tie2 activator.

Embodiment 36. The method of embodiment 35, wherein the cell is an ocular cell.

Embodiment 37. A method of administering a tyrosine phosphatase suppressor to a cell, the method comprising contacting a cell with a nucleic acid, wherein the nucleic acid is carried by a vector, wherein the nucleic acid encodes a tyrosine phosphatase suppressor.

Embodiment 38. The method of embodiment 37, wherein the cell is an ocular cell.

What is claimed is:

1. A pharmaceutical composition comprising a nucleic acid, wherein the nucleic acid is carried by a vector, wherein the nucleic acid encodes a tyrosine phosphatase suppressor.

2. The pharmaceutical composition of claim 1, wherein the tyrosine phosphatase is HPTPβ.

3. The pharmaceutical composition of claim 1, wherein the tyrosine phosphatase suppressor is a monoclonal antibody or an antigen-binding fragment thereof.

4. The pharmaceutical composition of claim 1, wherein the vector is a viral vector.

5. The pharmaceutical composition of claim 4, wherein the viral vector is an adenovirus-associated viral vector.

6. The pharmaceutical composition of claim 1, wherein the tyrosine phosphatase suppressor binds an extracellular domain of HPTPβ.

7. The pharmaceutical composition of claim 1, wherein the tyrosine phosphatase suppressor binds the first FN3 repeat of an extracellular domain of HPTPβ.

8. The pharmaceutical composition of claim 6, wherein the tyrosine phosphatase suppressor binds a sequence with at least 90% homology to SEQ ID NO.: 17.

9. The pharmaceutical composition of claim 3, wherein the monoclonal antibody or the antigen-binding fragment thereof comprises a heavy chain variable region having at least 90% homology to SEQ ID NO.: 1.

10. The pharmaceutical composition of claim 3, wherein the monoclonal antibody or the antigen-binding fragment thereof comprises a light chain variable region having at least 90% homology to SEQ ID NO.: 4.

11. The pharmaceutical composition of claim 3, wherein the monoclonal antibody or the antigen-binding fragment thereof comprises a heavy chain variable region and a light chain variable region, wherein the heavy chain variable region has at least 90% homology to SEQ ID NO.: 1 and the light chain variable region has at least 90% homology to SEQ ID NO.: 4.

12. The pharmaceutical composition of claim 1, the pharmaceutical composition further comprising a pharmaceutically-acceptable excipient, wherein the pharmaceutical composition is in a unit dosage form.

13. The pharmaceutical composition of claim 1, comprising from about 1 ng to about 1 mg of the vector.

14. A pharmaceutical composition comprising a nucleic acid, wherein the nucleic acid is carried by a vector, wherein the nucleic acid encodes a Tie2 activator.

15. The pharmaceutical composition of claim 14, wherein the vector is a viral vector.

16. The pharmaceutical composition of claim 15, wherein the viral vector is an adenovirus-associated viral vector.

17. The pharmaceutical composition of claim 14, wherein the Tie2 activator is a monoclonal antibody or an antigen-binding fragment thereof.

18. The pharmaceutical composition of claim 17, wherein the monoclonal antibody or antigen-binding fragment thereof binds to a tyrosine phosphatase.

19. The pharmaceutical composition of claim 18, wherein the tyrosine phosphatase is HPTPβ.

20. The pharmaceutical composition of claim 14, wherein the Tie2 activator binds an extracellular domain of HPTPβ.

21. The pharmaceutical composition of claim 14, wherein the Tie2 activator binds the first FN3 repeat of an extracellular domain of HPTPβ.

22. The pharmaceutical composition of claim 20, wherein the Tie2 activator binds a sequence with at least 90% homology to SEQ ID NO.: 17.

23. The pharmaceutical composition of claim 17, wherein the monoclonal antibody or the antigen-binding fragment thereof comprises a heavy chain variable region having at least 90% homology to SEQ ID NO.: 1.

24. The pharmaceutical composition of claim 17, wherein the monoclonal antibody or the antigen-binding fragment thereof comprises a light chain variable region having at least 90% homology to SEQ ID NO.: 4.

25. The pharmaceutical composition of claim 17, wherein the monoclonal antibody or the antigen-binding fragment thereof comprises a heavy chain variable region and a light chain variable region, wherein the heavy chain variable region has at least 90% homology to SEQ ID NO.: 1 and the light chain variable region has at least 90% homology to SEQ ID NO.: 4.

26. The pharmaceutical composition of claim 14, the pharmaceutical composition further comprising a pharmaceutically-acceptable excipient, wherein the pharmaceutical composition is in a unit dosage form.

27. The pharmaceutical composition of claim 14, comprising from about 1 ng to about 1 mg of the vector.


SEQUENCE LISTING (18 sequences)

SEQ ID NO: 1 - protein, 122 aa, Artificial Sequence
  Description of Artificial Sequence Synthetic polypeptide
EVQLVETGGGLVQPKGSMKLSCAASGFTFNANAMNWIRQAPGKGLEWVARIRTKSNNYATYYAGSVKDRFTISRDDAQNMLYLQMNDLKTEDTAMYYCVRDYYGSSAWITYWGQGTLVTVSA

SEQ ID NO: 2 - protein, 122 aa, Artificial Sequence
  Description of Artificial Sequence Synthetic polypeptide
EVQLVETGGGLVQPKGSMILSCAASGFTFNANAMNWIRQAPGKGLEWVARIRTKSNNYATYYAGSVKDRFTISRDDAQNMLYLQMNDLKTEDTAMYYCVRDYYGSSAWTTYWGQGTLVTVSA

SEQ ID NO: 3 - protein, 122 aa, Artificial Sequence
  Description of Artificial Sequence Synthetic polypeptide
EVQLVETGGGLAQPKGSMKLSCAASGFTFNANAMNWIRQAPGKGLEWVARIRTKSNNYATYYAGSVKDRFTISRDDAQNMLYLQMNDLKTEDTAMYYCVRDYYGSSAWITYWGQGTLVTVSA

SEQ ID NO: 4 - protein, 107 aa, Artificial Sequence
  Description of Artificial Sequence Synthetic polypeptide
DIVMTQSHKFMSTSVGDRVSITCKASQHVGTAVAWYQQKPDQSPKQLIYWASTRHTGVPDRFTGSGSGTDFTLTISNVQSEDLADYFCQQYSSYPFTFGSGTKLEIK

SEQ ID NO: 5 - protein, 107 aa, Artificial Sequence
  Description of Artificial Sequence Synthetic polypeptide
DIVMTQSHKFMSTSVGDRVSITCKASQHVGTAVAWYQQKPDQSPKQLIYWASTRHTGVPDRFTGSGSGSDFTLTISNVQSEDLADYFCQQYSSYPFTFGSGTKLEIK

SEQ ID NO: 6 - protein, 8 aa, Artificial Sequence
  Description of Artificial Sequence Synthetic peptide
GFTFNANA

SEQ ID NO: 7 - protein, 10 aa, Artificial Sequence
  Description of Artificial Sequence Synthetic peptide
IRTKSNNYAT

SEQ ID NO: 8 - protein, 13 aa, Artificial Sequence
  Description of Artificial Sequence Synthetic peptide
VRDYYGSSAWITY

SEQ ID NO: 9 - protein, 6 aa, Artificial Sequence
  Description of Artificial Sequence Synthetic peptide
QHVGTA

SEQ ID NO: 10 - protein, 9 aa, Artificial Sequence
  Description of Artificial Sequence Synthetic peptide
QQYSSYPFT

SEQ ID NO: 11 - DNA, 366 bp, Artificial Sequence
  Description of Artificial Sequence Synthetic polynucleotide
gaggtgcagcttgttgagactggtggaggattggtgcagcctaaagggtcaatgaaactctcatgtgcagcctctggattcaccttcaatgccaatgccatgaactggatccgccaggctccaggaaagggtttggaatgggttgctcgcataagaactaaaagtaataattatgcaacatattatgccggttcggtgaaagacaggttcaccatctccagagatgatgcacagaacatgctctatctgcaaatgaacgacttgaaaactgaggacacagccatgtattactgtgtgcgagattactacggtagtagcgcctggattacttactggggccaagggactctggtcactgtctctgca

SEQ ID NO: 12 - DNA, 321 bp, Artificial Sequence
  Description of Artificial Sequence Synthetic polynucleotide
gacattgtgatgacccagtctcacaaattcatgtccacatcagtaggagacagggtcagcatcacctgcaaggccagtcagcatgtgggtactgctgtagcctggtatcaacagaaaccagaccaatctcctaaacaactgatttactgggcatccacccggcacactggagtccctgatcgcttcacaggcagtggatctgggacagatttcactctcaccattagtaatgtgcagtctgaagacttggcagattatttctgtcagcaatacagcagttatccattcacgttcggctcggggacaaagttggaaataaaa

SEQ ID NO: 13 - protein, 1997 aa, Homo sapiens [sequence omitted]

SEQ ID NO: 14 - DNA, 6075 bp, Homo sapiens
gtctcctctggatcttaactactgagcgcaatgctgagccatggagccgggttggccttgtggatcacactgagcctgctgcagactggactggcggagccagagagatgtaacttcaccctggcggagtccaaggcctccagccattctgtgtctatccagtggagaattttgggctcaccctgtaactttagcctcatctatagcagtgacaccctgggggccgcgttgtgccctacctttcggatagacaacaccacatacggatgtaaccttcaagatttacaagcaggaaccatctataacttcaagattatttctctggatgaagagagaactgtggtcttgcaaacagatcctttacctcctgctaggtttggagtcagtaaagagaagacgacttcaaccggcttgcatgtttggtggactccttcttccggaaaagtcacctcatatgaggtgcaattatttgatgaaaataaccaaaagatacagggggttcaaattcaagaaagtacttcatggaatgaatacacttttttcaatctcactgctggtagtaaatacaatattgccatcacagctgtttctggaggaaaacgttctttttcagtttataccaatggatcaacagtgccatctccagtgaaagatattggtatttccacaaaagccaattctctcctgatttcctggtcccatggttctgggaatgtggaacgataccggctgatgctaatggataaagggatcctagttcatggcggtgttgtggacaaacatgctacttcctatgcttttcacgggctgtcccctggctacctctacaacctcactgttatgactgaggctgcagggctgcaaaactacaggtggaaactagtcaggacagcccccatggaagtctcaaatctgaaggtgacaaatgatggcagtttgacctctctaaaagtcaaatggcaaagacctcctggaaatgtggattcttacaatatcaccctgtctcacaaagggaccatcaaggaatccagagtattagcaccttggattactgaaactcactttaaagagttagtccccggtcgactttatcaagttactgtcagctgtgtctctggtgaactgtctgctcagaagatggcagtgggcagaacatttccagacaaagttgcaaacctggaggcaaacaataatggcaggatgaggtctcttgtagtgagctggtcgccccctgctggagactgggagcagtatcggatcctactcttcaatgattctgtggtgctgctcaacatcactgtgggaaaggaagaaacacagtatgtcatggatgacacggggctcgtaccgggaagacagtatgaggtggaagtcattgttgagagtggaaatttgaagaattctgagcgttgccaaggcaggacagtccccctggctgtcctccagcttcgtgtcaaacatgccaatgaaacctcactgagtatcatgtggcagacccctgtagcagaatgggagaaatacatcatttccctagctgacagagacctcttactgatccacaagtcactctccaaagatgccaaagaattcacttttactgacctggtgcctggacgaaaatacatggctacagtcaccagtattagtggagacttaaaaaattcctcttcagtaaaaggaagaacagtgcctgcccaagtgactgacttgcatgtggccaaccaaggaatgaccagtagtctgtttactaactggacccaggcacaaggagacgtagaattttaccaagtcttactgatccatgaaaatgtggtcattaaaaatgaaagcatctccagtgagaccagcagatacagcttccactctctcaagtccggcagcctgtactccgtggtggtaacaacagtgagtggagggatctcttcccgacaagtggttgtggagggaagaacagtcccttccagtgtgagtggagtaacggtgaacaattccggtcgtaatgactacctcagcgtttcctggctcgtggcgcccggagatgtggataactatgaggtaacattgtctcatgacggcaaggtggttcagtcccttgtcattgccaagtctgtcagagaatgttccttcagctccctcaccccaggccgcctctacaccgtgaccataactacaaggagtggcaagtatgaaaatcactccttcagccaagagcggacagtgcctgacaaagtccagggagtcagtgttagcaactcagccaggagtgactatttaagggtatcctgggtgcatgccactggagactttgatcactatgaagtcaccattaaaaacaaaaacaacttcattcaaactaaaagcattcccaagtcagaaaacgaatgtgtatttgttcagctagtccctggacggttgtacagtgtcactgttactacaaaaagtggacaatatgaagccaatgaacaagggaatgggagaacaattccagagcctgttaaggatctaacattgcgcaacaggagcactgaggacttgcatgtgacttggtcaggagctaatggggatgtcgaccaatatgagatccagctgctcttcaatgacatgaaagtatttcctccttttcaccttgtaaataccgcaaccgagtatcgatttacttccctaacaccaggccgccaatacaaaattcttgtcttgacgattagcggggatgtacagcagtcagccttcattgagggcttcacagttcctagtgctgtcaaaaatattcacatttctcccaatggagcaacagatagcctgacggtgaactggactcctggtgggggagacgttgattcctacacggtgtcggcattcaggcacagtcaaaaggttgactctcagactattcccaagcacgtctttgagcacacgttccacagactggaggccggggagcagtaccagatcatgattgcctcagtcagcgggtccctgaagaatcagataaatgtggttgggcggacagttccagcatctgtccaaggagtaattgcagacaatgcatacagcagttattccttaatagtaagttggcaaaaagctgctggtgtggcagaaagatatgatatcctgcttctaactgaaaatggaatccttctgcgcaacacatcagagccagccaccactaagcaacacaaatttgaagatctaacaccaggcaagaaatacaagatacagatcctaactgtcagtggaggcctctttagcaaggaagcccagactgaaggccgaacagtcccagcagctgtcaccgacctgaggatcacagagaactccaccaggcacctgtccttccgctggaccgcctcagagggggagctcagctggtacaacatctttttgtacaacccagatgggaatctccaggagagagctcaagttgacccactagtccagagcttctctttccagaacttgctacaaggcagaatgtacaagatggtgattgtaactcacagtggggagctgtctaatgagtctttcatatttggtagaacagtcccagcctctgtgagtcatctcagggggtccaatcggaacacgacagacagcctttggttcaactggagtccagcctctggggactttgacttttatgagctgattctctataatcccaatggcacaaagaaggaaaactggaaagacaaggacctgacggagtggcggtttcaaggccttgttcctggaaggaagtacgtgctgtgggtggtaactcacagtggagatctcagcaataaagtcacagcggagagcagaacagctccaagtcctcccagtcttatgtcatttgctgacattgcaaacacatccttggccatcacgtggaaagggcccccagactggacagactacaacgactttgagctgcagtggttgcccagagatgcacttactgtcttcaacccctacaacaacagaaaatcagaaggacgcattgtgtatggtcttcgtccagggagatcctatcaattcaacgtcaagactgtcagtggtgattcctggaaaacttacagcaaaccaatttttggatctgtgaggacaaagcctgacaagatacaaaacctgcattgccggcctcagaactccacggccattgcctgttcttggatccctcctgattctgactttgatggttatagtattgaatgccggaaaatggacacccaagaagttgagttttccagaaagctggagaaagaaaaatctctgctcaacatcatgatgctagtgccccataagaggtacctggtgtccatcaaagtgcagtcggccggcatgaccagcgaggtggttgaagacagcactatcacaatgatagaccgcccccctcctccacccccacacattcgtgtgaatgaaaaggatgtgctaattagcaagtcttccatcaactttactgtcaactgcagctggttcagcgacaccaatggagctgtgaaatacttcacagtggtggtgagagaggctgatggcagtgatgagctgaagccagaacagcagcaccctctcccttcctacctggagtacaggcacaatgcctccattcgggtgtatcagactaattattttgccagcaaatgtgccgaaaatcctaacagcaactccaagagttttaacattaagcttggagcagagatggagagcttaggtggaaaacgcgatcccactcagcaaaaattctgtgatggaccactgaagccacacactgcctacagaatcagcattcgagcttttacacagctctttgatgaggacctgaaggaattcacaaagccactctattcagacacatttttttctttacccatcactactgaatcagagcccttgtttggagctattgaaggtgtgagtgctggtctgtttttaattggcatgctagtggctgttgttgccttattgatctgcagacagaaagtgagccatggtcgagaaagaccctctgcccgtctgagcattcgtagggatcgaccattatctgtccacttaaacctgggccagaaaggtaaccggaaaacttcttgtccaataaaaataaatcagtttgaagggcatttcatgaagctacaggctgactccaactaccttctatccaaggaatacgaggagttaaaagacgtgggccgaaaccagtcatgtgacattgcactcttgccggagaatagagggaaaaatcgatacaacaatatattgccctatgatgccacgcgagtgaagctctccaatgtagatgatgatccttgctctgactacatcaatgccagctacatccctggcaacaacttcagaagagaatacattgtcactcagggaccgcttcctggcaccaaggatgacttctggaaaatggtgtgggaacaaaacgttcacaacatcgtcatggtgacccagtgtgttgagaagggccgagtaaagtgtgaccattactggccagcggaccaggattccctctactatggggacctcatcctgcagatgctctcagagtccgtcctgcctgagtggaccatccgggagtttaagatatgcggtgaggaacagcttgatgcacacagactcatccgccactttcactatacggtgtggccagaccatggagtcccagaaaccacccagtctctgatccagtttgtgagaactgtcagggactacatcaacagaagcccgggtgctgggcccactgtggtgcactgcagtgctggtgtgggtaggactggaacctttattgcattggaccgaatcctccagcagttagactccaaagactctgtggacatttatggagcagtgcacgacctaagacttcacagggttcacatggtccagactgagtgtcagtatgtctacctacatcagtgtgtaagagatgtcctcagagcaagaaagctacggagtgaacaagaaaaccccttgtttccaatctatgaaaatgtgaatccagagtatcacagagatccagtctattcaaggcattgagaatgtacctgaagagctcctggataaaaattattcactgtgtgatttgtt

SEQ ID NO: 15 - protein, 1998 aa, Mus musculus
MLRHGALTALWITLSVVQTGVAEQVKCNFTLLESRVSSLSASIQWRTFASPCNFSLIYSSDTSGPMWCHPIRIDNFTYGCNPKDLQAGTVYNFRIVSLDGEESTLVLQTDPLPPARFEVNREKTASTTLQVRWTPSSGKVSWYEVQLFDHNNQKIQEVQVQESTTWSQYTFLNLTEGNSYKVAITAVSGEKRSFPVYINGSTVPSPVKDLGISPNPNSLLISWSRGSGNVEQYRLVLMDKGAIVQDTNVDRRDTSYAFHELTPGHLYNLTIVTMASGLQNSRWKLVRTAPMEVSNLKVTNDGRLTSLNVKWQKPPGDVDSYSITLSHQGTIKESKTLAPPVTETQFKDLVPGRLYQVTISCISGELSAEKSAAGRTVPEKVRNLVSYNEIWMKSFTVNWTPPAGDWEHYRIVLFNESLVLLNTTVGKEETHYALDGLELIPGRQYEIEVIVESGNLRNSERCQGRTVPLAVLQLRVKHANETSLGITWRAPLGEWEKYIISLMDRELLVIHKSLSKDAKEFTFTDLMPGRNYKATVTSMSGDLKQSSSIKGRTVPAQVTDLHVNNQGMTSSLFTNWTKALGDVEFYQVLLIHENVVVKNESVSSDTSRYSFRALKPGSLYSVVVTTVSGGISSRQVVAEGRTVPSSVSGVTVNNSGRNDYLSVSWLPAPGEVDHYVVSLSHEGKVDQFLIIAKSVSECSFSSLTPGRLYNVTVTTKSGNYASHSFTEERTVPDKVQGISVSNSARSDYLKVSWVHATGDFDHYEVTIKNRESFIQTKTIPKSENECEFIELVPGRLYSVTVSTKSGQYEASEQGTGRTIPEPVKDLTLLNRSTEDLHVTWSRANGDVDQYEVQLLFNDMKVFPHIHLVNTATEYKFTALTPGRHYKILVLTISGDVQQSAFIEGLTVPSTVKNIHISANGATDRLMVTWSPGGGDVDSYVVSAFRQDEKVDSQTIPKHASEHTFHRLEAGAKYRIAIVSVSGSLRNQIDALGQTVPASVQGVVAANAYSSNSLTVSWQKALGVAERYDILLLNENGLLLSNVSEPATARQHKFEDLTPGKKYKMQILTVSGGLFSKESQAEGRTVPAAVTNLRITENSSRYLSFGWTASEGELSWYNIFLYNPDRTLQERAQVDPLVQSFSFQNLLQGRMYKMVIVTHSGELSNESFIFGRTVPAAVNHLKGSHRNTTDSLWFSWSPASGDFDFYELILYNPNGTKKENWKEKDVTEWRFQGLVPGRKYTLYVVTHSGDLSNKVTGEGRTAPSPPSLLSFADVANTSLAITWKGPPDWTDYNDFELQWFPGDALTIFNPYSSRKSEGRIVYGLHPGRSYQFSVKTVSGDSWKTYSKPISGSVRTKPDKIQNLHCRPQNSTAIACSWIPPDSDFDGYSIECRKMDTQEIEFSRKLEKEKSLLNIMMLVPHKRYLVSIKVQSAGMTSEVVEDSTITMIDRPPQPPPHIRVNEKDVLISKSSINFTVNCSWFSDTNGAVKYFAVVVREADSMDELKPEQQHPLPSYLEYRHNASIRVYQTNYFASKCAESPDSSSKSFNIKLGAEMDSLGGKCDPSQQKFCDGPLKPHTAYRISIRAFTQLFDEDLKEFTKPLYSDTFFSMPITTESEPLFGVIEGVSAGLFLIGMLVALVAFFICRQKASHSRERPSARLSIRRDRPLSVHLNLGQKGNRKTSCPIKINQFEGHFMKLQADSNYLLSKEYEDLKDVGRSQSCDIALLPENRGKNRYNNILPYDASRVKLCNVDDDPCSDYINASYIPGNNFRREYIATQGPLPGTKDDFWKMAWEQNVHNIVMVTQCVEKGRVKCDHYWPADQDPLYYGDLILQMVSESVLPEWTIREFKICSEEQLDAHRLIRHFHYTVWPDHGVPETTQSLIQFVRTVRDYINRSPGAGPTVVHCSAGVGRTGTFVALDRILQQLDSKDSVDIYGAVHDLRLHRVHMVQTECQYVYLHQCVRDVLRAKKLRNEQENPLFPIYENVNPEYHRDAIYSRH

SEQ ID NO: 16 - protein, 710 aa, Mus musculus
EQVKCNFTLLESRVSSLSASIQWRTFASPCNFSLIYSSDTSGPMWCHPIRIDNFTYGCNPKDLQAGTVYNFRIVSLDGEESTLVLQTDPLPPARFEVNREKTASTTLQVRWTPSSGKVSWYEVQLFDHNNQKIQEVQVQESTTWSQYTFLNLTEGNSYKVAITAVSGEKRSFPVYINGSTVPSPVKDLGISPNPNSLLISWSRGSGNVEQYRLVLMDKGAIVQDTNVDRRDTSYAFHELTPGHLYNLTIVTMASGLQNSRWKLVRTAPMEVSNLKVTNDGRLTSLNVKWQKPPGDVDSYSITLSHQGTIKESKTLAPPVTETQFKDLVPGRLYQVTISCISGELSAEKSAAGRTVPEKVRNLVSYNEIWMKSFTVNWTPPAGDWEHYRIVLFNESLVLLNTTVGKEETHYALDGLELIPGRQYEIEVIVESGNLRNSERCQGRTVPLAVLQLRVKHANETSLGITWRAPLGEWEKYIISLMDRELLVIHKSLSKDAKEFTFTDLMPGRNYKATVTSMSGDLKQSSSIKGRTVPAQVTDLHVNNQGMTSSLFTNWTKALGDVEFYQVLLIHENVVVKNESVSSDTSRYSFRALKPGSLYSVVVTTVSGGISSRQVVAEGRTVPSSVSGVTVNNSGRNDYLSVSWLPAPGEVDHYVVSLSHEGKVDQFLIIAKSVSECSFSSLTPGRLYNVTVTTKSGNYASHSFTEERTVP

SEQ ID NO: 17 - protein, 1621 aa, Homo sapiens
MLSHGAGLALWITLSLLQTGLAEPERCNFTLAESKASSHSVSIQWRILGSPCNFSLIYSSDTLGAALCPTFRIDNTTYGCNLQDLQAGTIYNFRIISLDEERTVVLQTDPLPPARFGVSKEKTTSTSLHVWWTPSSGKVTSYEVQLFDENNQKIQGVQIQESTSWNEYTFFNLTAGSKYNIAITAVSGGKRSFSVYTNGSTVPSPVKDIGISTKANSLLISWSHGSGNVERYRLMLMDKGILVHGGVVDKHATSYAFHGLTPGYLYNLTVMTEAAGLQNYRWKLVRTAPMEVSNLKVTNDGSLTSLKVKWQRPPGNVDSYNITLSHKGTIKESRVLAPWITETHFKELVPGRLYQVTVSCVSGELSAQKMAVGRTFPDKVANLEANNNGRMRSLVVSWSPPAGDWEQYRILLFNDSVVLLNITVGKEETQYVMDDTGLVPGRQYEVEVIVESGNLKNSERCQGRTVPLAVLQLRVKHANETSLSIMWQTPVAEWEKYIISLADRDLLLIHKSLSKDAKEFTFTDLVPGRKYMATVTSISGDLKNSSSVKGRTVPAQVTDLHVANQGMTSSLFTNWTQAQGDVEFYQVLLIHENVVIKNESISSETSRYSFHSLKSGSLYSVVVTTVSGGISSRQVVVEGRTVPSSVSGVTVNNSGRNDYLSVSWLLAPGDVDNYEVTLSHDGKVVQSLVIAKSVRECSFSSLTPGRLYTVTITTRSGKYENHSFSQERTVPDKVQGVSVSNSARSDYLRVSWVHATGDFDHYEVTIKNKNNFIQTKSIPKSENECVFVQLVPGRLYSVTVTTKSGQYEANEQGNGRTIPEPVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVTDLRITENSTRHLSFRWTASEGELSWYNIFLYNPDGNLQERAQVDPLVQSFSFQNLLQGRMYKMVIVTHSGELSNESFIFGRTVPASVSHLRGSNRNTTDSLWFNWSPASGDFDFYELILYNPNGTKKENWKDKDLTEWRFQGLVPGRKYVLWVVTHSGDLSNKVTAESRTAPSPPSLMSFADIANTSLAITWKGPPDWTDYNDFELQWLPRDALTVFNPYNNRKSEGRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKCDPTQQKFCDGPLKPHTAYRISIRAFTQLFDEDLKEFTKPLYSDTFFSLPITTESEPLFGAIE

SEQ ID NO: 18 - protein, 89 aa, Homo sapiens
LAEPERCNFTLAESKASSHSVSIQWRILGSPCNFSLIYSSDTLGAALCPTFRIDNTTYGCNLQDLQAGTIYNFRIISLDEERTVVLQTD